(19) United States
(12) Patent Application Publication
(10) Pub. No.: US 2025/0090466 A1
(43) Pub. Date: Mar. 20, 2025

(54) DRUG DELIVERY COMPOSITIONS AND DEVICES

(71) Applicant: Ohio State Innovation Foundation, Columbus, OH (US)

(72) Inventors: John LANNUTTI; Marco COUTINHO DA SILVA, Worthington, OH (US)

(21) Appl. No.: 18/727,296

(22) PCT Filed: Jan. 9, 2023
(86) PCT No.: PCT/US2023/060327

§ 371 Date: Jul. 8, 2024

Related U.S. Application Data
(60) Provisional application No. 63/297,433, filed on Jan. 7, 2022.

(51) Int. Cl. A61K 9/48; A61K 38/24

(52) U.S. Cl. CPC A61K 9/4816; A61K 9/4833; A61K 38/24

(57) ABSTRACT

Described herein are compositions and devices for the delivery of active agents. The composition for drug delivery comprising: a hydrophilic active agent encapsulated within a polysiloxane polymer; wherein the polysiloxane polymer comprises a hydrophilic polysiloxane polymer, an amphiphilic polysiloxane polymer, or any combination thereof.

### CROSS-REFERENCE TO RELATED APPLICATIONS

The application claims the benefit of U.S. Provisional Application No. 63/297,433, filed Jan. 7, 2022, which is hereby incorporated herein by reference in its entirety.

### BACKGROUND

Long-term delivery of drugs in an effective manner can be compromised by the fact that they may not remain biologically active. At 37° C. and in the presence of water many compounds are damaged by hydrolytic degradation, or the chemical reaction of water with specific locations of the chemical structure of the compound. Thus, the activity of the drug on day 1 may be 100% but after a year has passed may be reduced to 0% thus rendering the implant ineffective as a drug delivery vehicle.

Accordingly, there is a need for delivery vehicles for long term delivery of drugs that allow the drugs to retain their activity.

The compositions and methods disclosed herein address these and other needs.

### SUMMARY

Provided herein are compositions and devices for the delivery of active agents, particularly hydrophilic active agent. Described herein are compositions for drug delivery including: a hydrophilic active agent encapsulated within a polysiloxane polymer. The polysiloxane polymer can include a hydrophilic polysiloxane polymer, an amphiphilic polysiloxane polymer, or any combination thereof. In some embodiments, the polysiloxane polymer can further include a hydrophobic polysiloxane polymer.

In some embodiments, the hydrophilic active agent can include a gonodotropin releasing hormone (GnRh) agonist or pharmaceutically acceptable salt or prodrug thereof (e.g., deslorelin or pharmaceutically acceptable salt or prodrug thereof).

In some embodiments, the hydrophilic active agent can be present in the composition at a concentration of from 1 μg/ml to 100,000 μg/ml.

Described herein are also drug delivery capsules including two closed ends, a wall coaxially disposed around a chamber, and the composition described herein disposed within the chamber. In some embodiments, the wall can include a biodegradable polymer. In some embodiments, the wall can further include a non-biodegradable polymer.

In some embodiments, the wall can include a plurality of pores formed therewithin. In some embodiments, the pores can have an average pore size of from 100 nm to 5 μm. In some embodiments, the capsule can have a length of from 0.1 cm to 5 cm. In some embodiments, the chamber can have a cross-sectional diameter of from 100 μm to 5000 μm. In some embodiments, the wall can have a thickness of from 1 μm to 1,000 μm. In some embodiments, the active agent can be present in the composition in an amount of from 1 μg/ml to 100,000 μg/ml.

In some embodiments, the capsule can release the hydrophilic active agent over a period of at least 30 days, at least 3 months, at least 6 months, at least 9 months, or at least 12 months when incubated in phosphate buffered saline (PBS) at 37° C.

Described herein are also methods for preparing a drug delivery capsule, the method including: electrospinning a wall forming solution comprising a biodegradable polymer and a porogen to form a wall precursor having a lumen extending therethrough from a first end to a second end; removing the porogen from the wall precursor; sintering the wall precursor; injecting the composition described herein into the lumen of the wall precursor; and sealing the first end and the second end to form the drug delivery capsule.

In some embodiments, electrospinning the wall forming solution can include electrically charging the wall forming solution; and discharging the electrically charged wall forming solution onto a grounded target under an electrostatic field, such that the movement of the electrically charged wall forming solution under the electrostatic field causes the electrically charged wall forming solution to evaporate and produce fibers that form the wall precursor on the grounded target.

Described herein are also methods of contraceptive treatment in a subject in need thereof including administering to the subject an effective amount of an active agent using a drug delivery capsule described herein or a composition of described herein.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1A-1F: Microstructure of PCL:PET:HEPES under different conditions: 80:10:10 for IA) as-spun, 1B) water-treated fibers, 1C) sintered and 1D) water-treated post-sintering. Microstructure of sintered and water-treated PCL:PET:HEPES with varied HEPES content for comparison: 1E) 88:10:2 and 1F) 70:10:20.

FIG. 2: Water absorption of PCL:PET:HEPES capsules versus different amounts of removed salt content and oil carriers.

FIG. 3A-3B: Representative stress-elongation curves for 80:10:10 PCL:PET:HEPES: 3A) electrospun and 3B) post-sintering with and without salt leaching prior to tensile testing.

FIG. 4: Rose Bengal dye release from dense PCL:PET:HEPES (80:10:10) following salt removal using different carrier oils.

FIG. 5: Rose Bengal release from dense PCL:PET:HEPES ratios following HEPES removal using DBE-224 oil as the carrier.

FIG. 6A-6B: Simulated RB release from dense PCL:PET:HEPES with DBE-224 carrier oil at different salt ratios following salt removal: assuming 6A) interconnected porosity (Φ) is present and 6B) no interconnected porosity, both with a k=2,000.

FIG. 7A-7D: Cross-section microstructure of PCL:PET:HEPES at different salt concentrations: 7A) 5%, 7B) 10% and 7C) 20% post-leaching and 7D) 20% prior to leaching. The dark areas in 7D) represent HEPES embedded within the polymer matrix.

FIG. 8: Proposed saltatory motion of RB through a polymer matrix composed of PCL:PET containing HEPES generated porosity. Diffusion is approximately 4-6 orders of magnitude slower through the PCL:PET matrix (˜) than through the hydrated pores (→). Clearly, higher HEPES contents allow for a longer effective pathway for “fast diffusion” and, therefore, a greater release rate.

FIG. 9 shows the net results of capsule exposure of a dense polycaprolactone (PCL) capsule containing silicone oil (40 μL) to phosphate buffered saline (PBS) over a period of 50 days.

FIG. 10 shows the visual results of exposure of a contraceptive, deslorelin, to silicone oil alone at 37° C. What is apparent is that the deslorelin has some solubility within the oil.

FIG. 11 shows the net release from a dense polycaprolactone (PCL) capsule containing the hydrophilic silicone oil dimethylsiloxane-(60-70% ethylene oxide) block copolymer (DBE 712) and Rose Bengal into phosphate buffered saline (PBS) over a period of 64 days.

FIG. 12 shows four (4) mg of deslorelin powder (a) powder as-received (left hand side of image) and (b) following compaction in a metal die (right hand side of image).

FIG. 13 shows an exemplary drug delivery tubular capsule 100 including two closed ends 101a and 101b, a wall 102 coaxially disposed around a chamber 103, and a composition described herein disposed within the chamber 103.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

The term “comprising” and variations thereof as used herein is used synonymously with the term “including” and variations thereof and are open, non-limiting terms and do not exclude additional elements or steps. Although the terms “comprising” and “including” have been used herein to describe various embodiments, the terms “consisting essentially of” and “consisting of” can be used in place of “comprising” and “including” to provide for more specific embodiments of the invention and are also disclosed. For example, the terms “comprise” and/or “comprising,” when used in this specification, specify the presence of stated features, integers, steps, operations, elements, and/or components, but do not preclude the presence or addition of one or more other features, integers, steps, operations, elements, components, and/or groups thereof. Accordingly, these terms are intended to not only cover the recited element(s) or step(s), but may also include other elements or steps not expressly recited. Furthermore, as used herein, the use of the terms “a”, “an”, and “the” when used in conjunction with an element may mean “one,” but it is also consistent with the meaning of“one or more,” “at least one,” and “one or more than one.” Therefore, an element preceded by “a” or “an” does not, without more constraints, preclude the existence of additional identical elements. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraocular, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the terms “beneficial agent” and “active agent” are used interchangeably herein to refer to a natural or synthetically derived chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate”, “inactivating” and “inactivation” means to decrease or eliminate an activity, response, condition, disease, or other biological parameter due to a chemical (covalent bond formation) between the ligand and a its biological target.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, a symptom of a disease or disorder, or preventing or altering a physiological process. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event. In particular embodiments, “prevention” includes reduction in risk of coronavirus infection in patients. However, it will be appreciated that such prevention may not be absolute, i.e., it may not prevent all such patients developing a coronavirus infection, or may only partially prevent an infection in a single individual. As such, the terms “prevention” and “prophylaxis” may be used interchangeably.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial agent or agents can also refer to an amount covering both therapeutically effective amounts and prophylactically effective amounts. Dosage regimens can be adjusted to provide the optimum therapeutic response. For example, several divided doses may be administered daily or the dose may be proportionally reduced or increased as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

As used herein, by a “subject” is meant an individual. Thus, the “subject” can include companion or domesticated animals (e.g., cats, dogs, horses etc.), livestock (e.g., cattle, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

Reference will now be made in detail to specific aspects of the disclosed materials, compounds, compositions, articles, and methods, examples of which are illustrated in the accompanying Examples and Figures.

### Composition

Described herein are compositions for drug delivery including a hydrophilic active agent encapsulated within a polysiloxane polymer. The polysiloxane polymer can include a hydrophilic polysiloxane polymer, an amphiphilic polysiloxane polymer, or any combination thereof. In some embodiments, the composition can further include a hydrophobic polysiloxane polymer.

In some embodiments, the one or more hydrophilic active agent can be a solid. In some embodiments, the one or more hydrophilic active agent can be a powder.

In some embodiments, the hydrophilic active agent may include a gonodotropin releasing hormone (GnRh) agonist, antagonist, bioconjugate or pharmaceutically acceptable salts or prodrugs, thereof such as deslorelin, histrelin, avorelin, leuprolide, triptorelin, nafarelin, goserelin, buserelin, or fertirelin; or an immunocontraceptive agent based on zona pellucida (ZP) (i.e., porcine zona pellucida) or gonadotropin-releasing hormone (GnRH); or GnRH-based bioconjugates. In some embodiments, the gonodrotropin releasing hormone agonist can include deslorelin or pharmaceutically acceptable salt or prodrug thereof. In some embodiments, the hydrophilic active agent may include zona pellucida.

In some embodiments, the hydrophilic active agent can be present in the composition in a concentration of from 1 μg/ml to 100,000 μg/ml, (e.g., 1 μg/ml to 50,000 μg/ml, 1 μg/ml to 10,000 μg/ml, 1 μg/ml to 1,000 μg/ml, 1 μg/ml to 250 μg/ml, 1 μg/ml to 50 μg/ml, 1 μg/ml to 10 μg/ml, 1 μg/ml to 5 μg/ml, 10 μg/ml to 50,000 μg/ml, 10 μg/ml to 10,000 μg/ml, 10 μg/ml to 1,000 μg/ml, 10 μg/ml to 250 μg/ml, 10 μg/ml to 50 μg/ml, 50 μg/ml to 50,000 μg/ml, 50 μg/ml to 10,000 μg/ml, 50 μg/ml to 1,000 μg/ml, 50 μg/ml to 250 μg/ml, 250 μg/ml to 50,000 μg/ml, 250 μg/ml to 10,000 μg/ml, 250 μg/ml to 1,000 μg/ml, 1000 μg/ml to 50,000 μg/ml, 1000 μg/ml to 10,000 μg/ml, or 10000 μg/ml to 50,000 μg/ml).

In some embodiments, the polysiloxane polymer can be any suitable hydrophilic polysiloxane polymer such as dimethylsiloxane-ethylene oxide block/graft co polymers (e.g., dimethylsiloxane-(25-30% ethylene oxide) block copolymer, dimethylsiloxane-(30-35% ethylene oxide) block copolymer, dimethylsiloxane-(45-50% ethylene oxide) block copolymer, dimethylsiloxane-(50-55% ethylene oxide) block copolymer, dimethylsiloxane-(60-70% ethylene oxide) block copolymer, dimethylsiloxane-acetoxy terminated ethylene oxide block copolymer, dimethylsiloxane-(80% ethylene oxide) block copolymer, dimethylsiloxane-(80-85% ethylene oxide) block copolymer, dimethylsiloxane-(85-90% ethylene oxide) block copolymer), (carbinol functional) methylsiloxane dimethylsiloxane copolymer, (hydroxypropyleneoxypropyl)methylsiloxane-dimethylsiloxane copolymer, hydroxyalkyl terminated poly(propyleneoxy)-polydimethylsiloxane block copolymer, (hydroxyethyleneoxypropylmethylsiloxane)-(3,4-dimethoxyphenylpropyl)methylsiloxane-dimethylsiloxane terpolymer, carbinol (hydroxyl) terminated polydimethylsiloxane, (35% hydroxyethyleneoxypropylmethylsiloxane)-(dimethylsiloxane) copolymer, carboxylate substituted (n-pyrrolidonepropyl)methylsiloxane-dimethylsiloxane copolymers, dimethylaminopropylcarboxamide substituted (n-pyrrolidonepropyl)methylsiloxane-dimethylsiloxane copolymers, or tetrahydrofurfuryloxypropylmethylsiloxane, cyanopropylmethylsiloxane, or amphiphilic polysiloxane polymer such as dodecylmethylsiloxane-hydroxypolyalkyleneoxypropylmethylsiloxane copolymer.

In some embodiments, the hydrophobic polysiloxane polymer can be represented by:

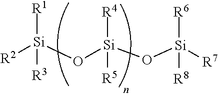

wherein
R¹-R⁸ are each independently alkyl, alkenyl, cycloalkyl, or aryl;
n is 1-500.

In some embodiments, the hydrophobic polysiloxane polymer can include, but is not limited to, polydimethylsiloxane, polydiethylsiloxane, polydipropylsiloxane, or polydiphenylsiloxane.

### Drug Delivery Capsule

Described herein are drug delivery capsules including two closed ends, a wall coaxially disposed around a chamber, and a composition described herein disposed within the chamber. In some embodiments, the capsules can have any suitable geometry including, but not limited to, tubular, cylindrical, hexagonal, square, square tubular, hexagonal tubular, cylindrical tubular. In some embodiments, the capsule can be tubular.

An exemplary drug delivery capsule 100 is shown in FIG. 13. The drug delivery capsule 100 can include two closed ends 101a and 101b, a wall 102 coaxially disposed around a chamber 103, and a composition described herein disposed within the chamber 103.

In some embodiments, the wall can include a biodegradable polymer.

In some embodiments, the biodegradable polymer can include a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof. In some embodiments, natural biodegradable materials (collagen, gelatin, etc.) may be partially or completely crosslinked, e.g., by exposure to glutaraldehyde vapor. In some embodiments, the biodegradable polymer can include polycaprolactone (PCL).

In some embodiments, the wall further includes a non-biodegradable polymer. In some embodiments, the non-biodegradable polymer can include polyethylene terephthalate (PET), polyvinyl chloride (PVC), polyethylene (PE), polysulfone (PSU), polyethersulfone (PES), polypropylene (PP), polystyrene (PS), poly(urethanes), poly(acrylates), poly(ethylene vinyl acetate), nylon, copolymers, or blends thereof. In some embodiments, the non-biodegradable polymer can include polyethylene terephthalate (PET).

In some embodiments, the capsule can have a length of at least 0.1 cm, (e.g., at least 0.5 cm, at least 1 cm, at least 1.5 cm, at least 2 cm, at least 2.5 cm, at least 3 cm, at least 3.5 cm, at least 4 cm, or at least 4.5 cm).

In some embodiments, the capsule can have a length of 5 cm or less, (e.g., 4.5 cm or less, 4 cm or less, 3.5 cm or less, 3 cm or less, 2.5 cm or less, 2 cm or less, 1.5 cm or less, 1 cm or less, or 0.5 cm or less).

The capsule can have a length ranging from any of the minimum values described above to any of the maximum values described above. For example, in some embodiments, the capsule can have a length of from 0.1 cm to 5 cm, such as from 0.5 cm to 2 cm, from 0.1 cm to 0.5 cm, from 0.1 cm to 1 cm, from 0.1 cm to 2 cm, from 0.1 cm to 3 cm, from 0.1 cm to 4 cm, from 0.5 cm to 2 cm, from 0.5 cm to 4 cm, from 0.5 cm to 5 cm, from 0.5 cm to 3 cm, from 1 cm to 2 cm, from 1 cm to 4 cm, from 1 cm to 5 cm, or from 1 cm to 3 cm.

In some embodiments, the chamber can have a cross-sectional diameter of at least 100 μm, (e.g., at least 250 μm, at least 500 μm, at least 750 μm, at least 1000 μm, at least 1250 μm, at least 1500 μm, at least 1750 μm, at least 2000 μm, at least 2250 μm, at least 2500 μm, at least 2750 μm, at least 3000 μm, at least 3250 μm, at least 3500 μm, at least 3750 μm, at least 4000 μm, at least 4250 μm, at least 4500 μm, or at least 4750 μm).

In some embodiments, the chamber can have a cross-sectional diameter of 5000 μm or less, (e.g., 4750 μm or less, 4500 μm or less, 4250 μm or less, 4000 μm or less, 3750 μm or less, 3500 μm or less, 3250 μm or less, 3000 μm or less, 2750 μm or less, 2500 μm or less, 2250 μm or less, 2000 μm or less, 1750 μm or less, 1500 μm or less, 1250 μm or less, 1000 μm or less, 750 μm or less, 500 μm or less, or 250 μm or less).

The capsule can have a length ranging from any of the minimum values described above to any of the maximum values described above. For example, in some embodiments, the chamber can have a cross-sectional diameter of from 100 μm to 5000 μm, such as from 500 μm to 4000 μm, from 500 μm to 3000 μm, from 500 μm to 2000 μm, from 500 μm to 5000 μm, from 500 μm to 1000 μm, from 200 μm to 1000 μm, from 200 μm to 2000 μm, from 200 μm to 3000 μm, from 200 μm to 4000 μm, from 200 μm to 5000 μm, from 200 μm to 600 μm, from 100 μm to 500 μm, from 100 μm to 200 μm, from 100 μm to 300 μm, from 100 μm to 400 μm, from 100 μm to 600 μm, from 100 μm to 700 μm, from 100 μm to 800 μm, from 100 μm to 900 μm, from 100 μm to 1000 μm, from 100 μm to 2000 μm, from 100 μm to 3000 μm, or from 100 μm to 4000 μm.

In some embodiments, the wall can have a thickness of at least 1 μm, (e.g., at least 5 μm, at least 10 μm, at least 25 μm, at least 50 μm, at least 75 μm, at least 100 μm, at least 150 μm, at least 200 μm, at least 250 μm, at least 300 μm, at least 350 μm, at least 400 μm, at least 450 μm, at least 500 μm, at least 550 μm, at least 600 μm, at least 650 μm, at least 700 μm, at least 750 μm, at least 800 μm, at least 850 μm, at least 900 μm, or at least 950 μm).

In some embodiments, the wall can have a thickness of 1000 μm or less, (e.g., 950 μm or less, 900 μm or less, 850 μm or less, 800 μm or less, 750 μm or less, 700 μm or less, 650 μm or less, 600 μm or less, 550 μm or less, 500 μm or less, 450 μm or less, 400 μm or less, 350 μm or less, 300 μm or less, 250 μm or less, 200 μm or less, 150 μm or less, 100 μm or less, 75 μm or less, 50 μm or less, 25 μm or less, 10 μm or less, or 5 μm or less).

The wall can have a thickness ranging from any of the minimum values described above to any of the maximum values described above. For example, in some embodiments, the wall can have a thickness of from 1 μm to 1,000 μm (e.g., from 1 μm to 500 μm, from 1 μm to 750 μm, from 1 μm to 250 μm, from 1 μm to 100 μm, from 1 μm to 50 μm, from 1 μm to 10 μm, from 1 μm to 5 μm, from 50 μm to 100 μm, from 50 μm to 200 μm, from 50 μm to 500 μm, from 50 μm to 1000 μm, from 100 μm to 200 μm, from 100 μm to 500 μm, from 100 μm to 1,000 μm, from 500 μm to 750 μm, from 500 μm to 1,000 μm, from 10 μm to 50 μm, from 10 μm to 100 μm, from 10 μm to 500 μm, or from 10 μm to 1000 μm).

The wall can have a porosity of at least 5% as determined by mercury porosimetry or apparent density (e.g., at least 10%, at least 20%, at least 30%, at least 40%, at least 50%, or at least 60%). The wall can have a porosity of 70% or less as determined by mercury porosimetry or apparent density (e.g., 60% or less, 50% or less, 40% or less, 30% or less, 20% or less, or 10% or less).

The wall can have a porosity ranging from any of the minimum values described above to any of the maximum values described above. For example, in some embodiments, the wall can have a porosity of from 5% to 70% as determined by mercury porosimetry or apparent density (e.g., from 5% to 60%, from 5% to 50%, from 5% to 40%, from 5% to 30%, from 5% to 20%, from 5% to 10%, from 10% to 70%, from 10% to 60%, from 10% to 50%, from 10% to 40% from 10% 30%, from 10% to 20%, from 20% to 30%, from 20% to 40%, from 20% to 50%, from 20% to 60%, from 20% to 70%, from 30% to 70%, from 30% to 60%, from 30% to 50%, from 30% to 40%, from 40% to 70%, from 40% to 60% from 40% to 50%, from 50% to 60%, from 50% 70%, or from 60% to 70%).

The wall can have a density of at least 0.25 g/c as determined by mercury porosimetry or apparent density (e.g., at least 0.35 g/c, at least 0.45 g/c, or at least 0.65 g/c). The wall can have a density of 0.70 g/c or less as determined by mercury porosimetry or apparent density (e.g., 0.65 g/c or less, 0.60 g/c or less, 0.55 g/c or less, 0.50 g/c or less, 0.45 g/c or less, 0.40 g/c or less, 0.35 g/c or less, or 0.30 g/c or less).

The wall can have a density ranging from any of the minimum values described above to any of the maximum values described above. For example, in some embodiments, the wall can have a density of from 0.25 g/c to 0.70 g/c as determined by mercury porosimetry or apparent density, (e.g., from 0.25 g/c to 0.60 g/c, from 0.25 g/c to 0.50 g/c, from 0.25 g/c to 0.40 g/c, from 0.25 g/c to 0.30 g/c, from 0.3 g/c to 0.60 g/c, from 0.3 g/c to 0.50 g/c, from 0.3 g/c to 0.40 g/c, from 0.35 g/c to 0.60 g/c, from 0.35 g/c to 0.50 g/c, from 0.35 g/c to 0.40 g/c, from 0.4 g/c to 0.60 g/c, from 0.4 g/c to 0.50 g/c, from 0.50 g/c to 0.60 g/c, from 0.50 g/c to 0.70 g/c, from 0.40 g/c to 0.70 g/c, or from 0.30 g/c to 0.70 g/c).

The wall can have a porosity of at least 5% (e.g., at least 10%, at least 20%, at least 30%, at least 40%, at least 50%, or at least 60%) and a density of at least 0.25 g/c (e.g., at least 0.35 g/c, at least 0.45 g/c, or at least 0.65 g/c) as determined by mercury porosimetry or apparent density.

The wall can have a porosity of 70% or less (e.g., 60% or less, 50% or less, 40% or less, 30% or less, 20% or less, or 10% or less) and a density of 0.70 g/c or less (e.g., 0.65 g/c or less, 0.60 g/c or less, 0.55 g/c or less, 0.50 g/c or less, 0.45 g/c or less, 0.40 g/c or less, 0.35 g/c or less, or 0.30 g/c or less) as determined by mercury porosimetry or apparent density.

The wall can have a porosity and a density ranging from any of the minimum values described above to any of the maximum values described above. For example, in some embodiments, the tubular matrix can have a porosity of from 5% to 70% (e.g., from 5% to 60%, from 5% to 50%, from 5% to 40%, from 5% to 30%, from 5% to 20%, from 5% to 10%, from 10% to 70%, from 10% to 60%, from 10% to 50%, from 10% to 40% from 10% 30%, from 10% to 20%, from 20% to 30%, from 20% to 40%, from 20% to 50%, from 20% to 60%, from 20% to 70%, from 30% to 70%, from 30% to 60%, from 30% to 50%, from 30% to 40%, from 40% to 70%, from 40% to 60% from 40% to 50%, from 50% to 60%, from 50% 70%, or from 60% to 70%) and a density of from 0.25 g/c to 0.70 g/c, (e.g., from 0.25 g/c to 0.60 g/c, from 0.25 g/c to 0.50 g/c, from 0.25 g/c to 0.40 g/c, from 0.25 g/c to 0.30 g/c, from 0.3 g/c to 0.60 g/c, from 0.3 g/c to 0.50 g/c, from 0.3 g/c to 0.40 g/c, from 0.35 g/c to 0.60 g/c, from 0.35 g/c to 0.50 g/c, from 0.35 g/c to 0.40 g/c, from 0.4 g/c to 0.60 g/c, from 0.4 g/c to 0.50 g/c, from 0.50 g/c to 0.60 g/c, from 0.50 g/c to 0.70 g/c, from 0.40 g/c to 0.70 g/c, or from 0.30 g/c to 0.70 g/c) as determined by mercury porosimetry or apparent density.

In some embodiments, the wall can include a plurality of pores formed therewithin.

In some embodiments, the pores can have an average pore size of at least 100 nm, (e.g., at least 200 nm, at least 500 nm, at least 0.1 μm, at least 0.5 μm, at least 1 μm, at least 2 μm, at least 3 μm, or at least 4 μm).

In some embodiments, the pores can have an average pore size of 5 μm or less, (e.g., 4 μm or less, 3 μm or less, 2 μm or less, 1 μm or less, 0.5 μm or less, 0.1 μm or less, 500 nm or less, 250 nm or less, 200 nm or less, or 150 nm or less).

The wall can have a thickness ranging from any of the minimum values described above to any of the maximum values described above. For example, in some embodiments, the pores can have an average pore size of from 100 nm to 5 μm, such as from 100 nm to 2.5 μm, from 100 nm to 1 μm, from 100 nm to 0.1 μm, from 100 nm to 500 nm, from 100 nm to 250 nm, from 250 nm to 500 nm, from 250 nm to 2.5 μm, from 250 nm to 1 μm, from 250 nm to 0.1 μm, from 500 nm to 0.1 μm, from 500 nm to 0.5 μm, from 500 nm to 1 μm, from 500 nm to 3 μm, from 500 nm to 4 μm, from 500 nm to 5 μm, from 0.1 μm to 0.5 μm, from 0.1 μm to 1 μm, from 0.1 μm to 2 μm, from 0.1 μm to 3 μm, from 0.1 μm to 4 μm, from 0.1 μm to 5 μm, from 1 μm to 2.5 μm, from 1 μm to 5 μm, from 1.5 μm to 5 μm, from 2.5 μm to 5 μm, from 100 nm to 2 μm, or from 500 nm to 2 μm.

In some embodiments, the drug delivery capsule can release the active agent over a period of at least 30 days, (e.g., at least 3 months, at least 6 months, at least 9 months, or at least 12 months) when incubated in phosphate buffered saline (PBS) at 37° C.

In some embodiments, the drug delivery capsule can release the active agent over a period of 12 months or less, (e.g., 9 months or less, 6 months or less, 3 months or less, or 1.5 months or less) when incubated in phosphate buffered saline (PBS) at 37° C.

The drug delivery capsule can release the active agent over a period ranging from any of the minimum values described above to any of the maximum values described above. For example, in some embodiments, the drug delivery capsule can release the active agent over a period of from 30 days to 12 months, (e.g., from 30 days to 9 months, from 30 day to 6 months, from 30 days to 3 months, from 3 days to 9 months, from 3 day to 6 months, from 6 days to 9 months, from 6 days to 12 months, or from 9 day to 12 months).

### Methods of Making

Disclosed herein are also methods for preparing a drug delivery capsule, the method including electrospinning a wall forming solution including a biodegradable polymer and a porogen to form a wall precursor having a lumen extending therethrough from a first end to a second end; removing the porogen from the wall precursor; sintering the wall precursor; sealing the first end; injecting the composition described herein into the lumen of the wall precursor; and sealing the second end to form the drug delivery capsule.

In some embodiments, electrospinning the wall forming solution can include electrically charging the wall forming solution; and discharging the electrically charged wall forming solution onto a grounded target under an electrostatic field, such that the movement of the electrically charged wall forming solution under the electrostatic field causes the electrically charged wall forming solution to evaporate and produce fibers that form the wall precursor on the grounded target. In some embodiments, the grounded target can include a rotating mandrel.

In some embodiments, sintering can include at a temperature from 50° C. to 150° C., for example from 90° C. to 110° C. In some embodiments, sintering can include heating for a period from 1 minute to 6 hours, for example from 30 minutes to 6 hours, from 30 minutes to 3 hours, or from 1 hour to 4 hours.

In some embodiments, removing the porogen can include washing the drug delivery capsule following sintering. In some embodiments, the drug delivery capsule can be washed with a saturated sodium bicarbonate solution followed by deionized water. In some embodiments, the porogen can be substantially removed from the drug delivery capsule upon washing with deionized water.

In some embodiments, the capsule can be formed using standard extrusion techniques that involve formation of a high temperature polymer melt. This melt can then be carefully extruded to form tubes having either dense or porous walls made up of the same polymers that were previously electrospun. The extrusion process could also include a dissolvable porogen such that porous pathways could again be formed by extraction of the porogen by exposure to water, for example.

In some embodiments, the method can further include drying the drug delivery capsule following washing. In some embodiments, drying is in vacuo. In some embodiments, drying can be at a temperature of about 50° C. to about 150° C., for example from about 90° C. to 110° C. In some embodiments, drying occurs for a period from about 1 minute to about 6 hours, for example from about 30 minutes to about 6 hours.

In some aspects, the two ends of the capsule are closed. The ends may be closed by any number of sealing techniques as would be appropriately selected by one of skill in the art. In some embodiments, the two ends are sealed using a high frequency tube sealing technique. In such techniques, a high frequency generates an eddy current in the wall, which heats up at least the polymer layers. When the temperature has reached the melting point of the polymer, clamps are closed and the melted polymer is cooled and formed. In some embodiments, the two ends are sealed using hot-jaw tube sealing, where heated jaws apply heat to the outside of the tubular shape to heat up the inside for sealing. In some embodiments, the two ends may be sealed using ultrasonic tube sealing. In such techniques, the polymer composition of the inner layers is heated and melted by high frequency friction force introduced form an ultrasonic horn. Clamps are then closed around the section intended to be sealed, cooled, and formed to seal the ends. In some embodiments, the two ends are sealed using hot air sealing, wherein the system heats the seal area inside the capsule with hot air and then subsequently presses and chills the ends in a subsequent station.

In some embodiments, the biodegradable polymer can include a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof. In some embodiments, natural biodegradable materials (collagen, gelatin, etc.) may be partially or completely crosslinked, e.g., by exposure to glutaraldehyde vapor. In some embodiments, the biodegradable polymer can include polycaprolactone (PCL).

In some embodiments, the wall can further include a non-biodegradable polymer. In some embodiments, the non-biodegradable polymer can include polyethylene terephthalate (PET), polyvinyl chloride (PVC), polyethylene (PE), polysulfone (PSU), polyethersulfone (PES), polypropylene (PP), polystyrene (PS), poly(urethanes), poly(acrylates), poly(ethylene vinyl acetate), nylon, copolymers, or blends thereof. In some embodiments, the non-biodegradable polymer can include polyethylene terephthalate (PET).

In some embodiments, the biodegradable polymer, the non-biodegradable polymer, and porogen are present. In some embodiments, the biodegradable polymer, the non-biodegradable polymer, and porogen are present in a ratio of from 70:10:20 to 88:10:2 such as 70:10:20, 80:10:10, 82:10:8, 85:10:5, or 88:10:2 in the solution.

A “porogen” as used herein refers to any material that can be used to create a porous material, e.g. porous polycaprolactone as described herein. In some embodiments, the porogen comprises a water-soluble compound, i.e. such that the porogen is substantially removed from the outer layer upon washing the drug delivery device with water. In some embodiments, the porogen can include a soluble organic salt such as HEPES salt; biocompatible soluble inorganic salts such as NaCl or KCl; or any combination thereof. In some embodiments, the porogen can include a compound selected from ([Tris(hydroxymethyl)methylamino]propanesulfonic acid) (TAPS), (2-(Bis(2-hydroxyethyl)amino)acetic acid) (Bicine), (Tris(hydroxymethyl)aminomethane) or, (2-Amino-2-(hydroxymethyl)propane-1,3-diol) (Tris), (N-[Tris(hydroxymethyl)methyl]glycine) (Tricine), (3-[N-Tris(hydroxymethyl)methylamino]-2-hydroxypropanesulfonic acid) (TAPSO), (4-(2-hydroxyethyl)-1-piperazineethanesulfonic acid) (HEPES), (2-[[1,3-dihydroxy-2-(hydroxymethyl)propan-2-yl]amino]ethanesulfonic acid) (TES), (3-(N-morpholino)propanesulfonic acid) (MOPS), (Piperazine-N,N′-bis(2-ethanesulfonic acid)) (PIPES), Dimethylarsenic acid, (2-(N-morpholino)ethanesulfonic acid) (MES), or salts thereof, such as the sodium salts thereof.

In other embodiments, the disclosed capsules may be manufactured by any appropriate method as would be readily understood by those of ordinary skill in the art. In some embodiments, the disclosed capsules may be manufactured by asymmetric membrane formation; a representative example of such methods are provided in Yen, C. et al. “Synthesis and characterization of nanoporous polycaprolactone membranes via thermally- and nonsolvent-induced phase separations for biomedical device application” Journal of Membrane Science 2009, 343:180-88, hereby incorporated herein by reference in its entirety for all purposes. In some embodiments, the disclosed capsules may be manufactured using three-dimensional printing. In some embodiments, the disclosed capsules may be manufactured around methylcellulose which is subsequently removed to form the luminal compartment. In some embodiments, the disclosed capsules may be manufactured by a method described by Envisia Therapeutics in WO 2015/085251, WO 2016/144832, WO 2016/196365, WO 2017/015604, WO 2017/015616, or WO 2017/015675, each of which is hereby incorporated by reference in its entirety for all purposes. In yet other embodiments, the disclosed capsules may be manufactured by methods similar to those used in the manufacturing of hollow fiber membranes, such as phase inversion including non-solvent induced phase inversion (NIPS), (solvent) evaporation-induced phase inversion (EIPS), vapor sorption-induced phase inversion (VIPS), and thermally induced phase inversion (TIPS) In some embodiments, the disclosed capsules may be manufacturing using a method similar to the methods described in US 2015/232506, incorporated herein by reference in its entirety for all purposes. In some embodiments, the pores may instead by formed by laser diffraction of the capsules.

### Methods of Use

Described are methods of treating a clinical condition by administration to a subject in need thereof of an effective amount of an active agent using a composition and/or drug delivery capsule described herein. A clinical condition can be a clinical disorder, disease, dysfunction or other condition that can be ameliorated by a therapeutic composition.

Described herein are methods for contraceptive treatment in a subject in need thereof, the method including administering to the subject an effective amount of an active agent described herein using a composition and/or a drug delivery capsule described herein.

Described herein are also methods for controlling reproductive processes in a subject in need thereof, the method including administering to the subject an effective amount of an active agent described herein using a composition and/or a drug delivery capsule described herein.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the methods described herein without departing from the concept, spirit and scope of the disclosure. More specifically, it will be apparent that certain agents which are both chemically related may be substituted for the agents described herein while the same or similar results would be achieved. All such similar substitutes and modifications apparent to those skilled in the art are deemed to be within the spirit, scope and concept of the disclosure as defined by the appended claims.

By way of non-limiting illustration, examples of certain embodiments of the present disclosure are given below.

### EXAMPLES

### Example 1: Use of Hydrophobic Silicone Oil as a Drug Carrier for Long-Term Protection/Delivery of Moisture-Sensitive Compounds

The enhanced preservation and release of water-sensitive compounds contained within implanted capsules was identified and tested. A polymer-based capsule in which a desired drug compound is contained. Long-term delivery of such drugs in an effective manner can be compromised by the fact that they may not remain biologically active. At 37° C. and in the presence of water many such compounds are damaged by hydrolytic degradation, or the chemical reaction of water with specific locations of the chemical structure of the compound. Thus, the activity of the drug on day 1 may be 100% but after a year has passed may be reduced to 0% thus rendering the implant ineffective as a drug delivery vehicle.

The fact that many drugs display reasonable resistance to temperature, meaning that 37° C. exposure alone may not degrade their biological efficacy. However, removal of water from this internal environment is not possible if the carrier liquid within the capsule has an aqueous basis. This is also a limitation of many biodegradable polymers: their degradation is triggered by an inherent affinity for moisture, meaning that they normally would be poor hosts to a hydrolytically sensitive drug. Many non-aqueous solvents could be considered but are too toxic to be considered as well or have the potential to denature or degrade the compound of interest that also eliminates effectiveness.

The use of hydrophobic oils, particularly silicone oil, as a means of eliminating hydrolytic degradation of a compound contained within a capsule, are attractive. Such compounds could be either small or very large biologicals encompassing a broad range of intended medical purposes. In this context, these compounds may or may not be soluble in the oil of interest. They can exist as a solid if kept dry that retains its effectiveness upon release by the capsule through capsule rupture. If the drug has at least limited dissolution within the oil, transport through the surrounding hydrophobic liquid and then through the surrounding polymer wall of the capsule could occur.

Such a silicone-oil based environment can also prove useful in protecting electronic components from the deleterious effects of continuous water exposure that otherwise characterizes the biological environment and known biodegradable polymers under normal circumstances. Corrosion of delicate electronic connections could render such devices useless. Packaging them in an inert silicone oil environment allows for very rapid oxygen exchange while preventing/eliminating moisture, thus preventing moisture-based attack on the components. Conversely, if oxygen needs to be excluded from the use of an antioxidant compound or compounds into the oil can also be considered.

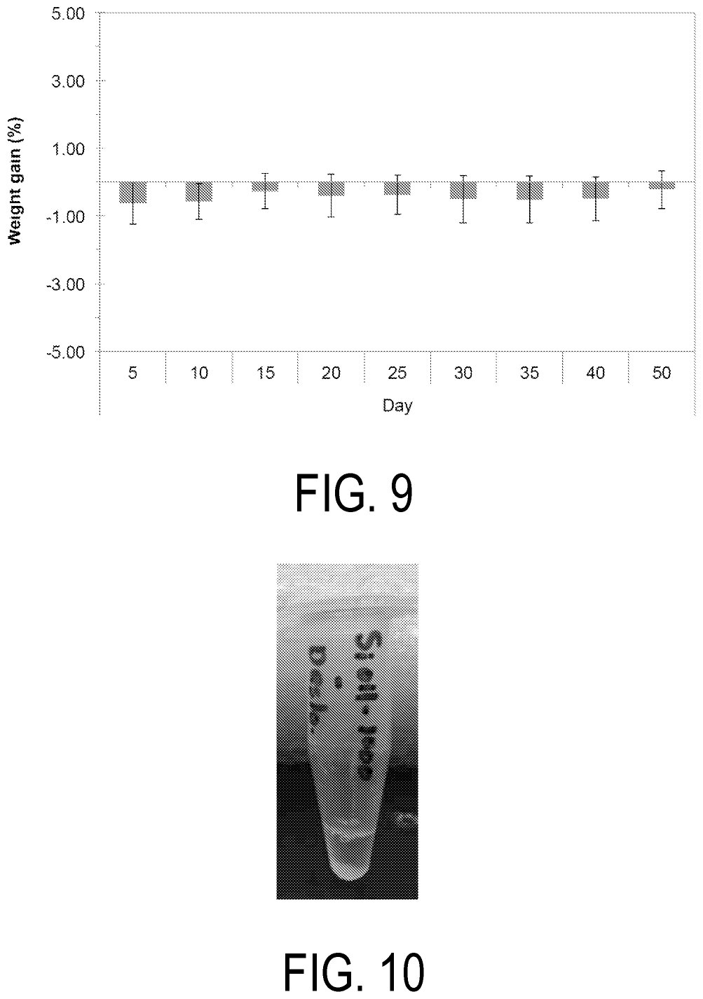

FIG. 9 shows the net results of capsule exposure of a dense polycaprolactone (PCL) capsule containing silicone oil (40 μL) to phosphate buffered saline (PBS) over a period of 50 days. Note that no weight gain is observed, indicating that no water enters the capsule. Thus, any drug contained within this capsule will be protected from hydrolytic degradation indefinitely.

FIG. 10 shows the visual results of exposure of a contraceptive, deslorelin, to silicone oil alone at 37° C. What is apparent is that the deslorelin has some solubility within the oil. This creates a solid ‘depot’ approach to drug release in which the capsule can continue to release the compound of interest through a surrounding wall via diffusion. The concentration of dissolved drug in the oil is then replenished by a corresponding dissolution of a small amount of any remaining solid. This process can occur until the last of the solid form of the drug dissolves. If the desired rates of drug release are within the 10-30 μg/day range, this can potentially allow for years of continuous release of a bioactive drug through the capsule wall.

This can have multiple applications in drug release applications that require years of continuous, low-level release of highly biologically active compounds. Contraception in mammals is one such target. Chemotherapeutic release is another potential application in which the bioactivity of the compound must be preserved, potentially for a period of years.

### Example 2: Use of Hydrophilic Silicone Oils to Enhance Release from Capsules Containing a Drug Surrounded by Hydrophobic Silicone Oil

We identified and tested a concept that will allow for the enhanced dissolution and release of water-sensitive compounds contained within implanted capsules containing silicone oil as a means of preserving bioactivity. This bioactivity could be focused on contraception or other drug delivery needs. This invention begins with a polymer-based capsule in which a desired drug compound is contained. Long-term delivery of such drugs in vivo in an effective manner can be compromised by the fact that they may not remain biologically active. At 37° C. and in the presence of water, many such compounds are damaged by hydrolytic degradation or the chemical reaction of water with specific chemical groups or moieties present within the compound. Thus, the activity of the drug on day 1 may be 100% but by the time a year has passed this bioactivity may be reduced to 0% thus rendering the implant ineffective as a drug delivery vehicle.

Removal of water from this internal environment is possible if the carrier liquid within the capsule is hydrophobic. This can be achieved using hydrophobic liquids such as silicone oil (polydimethylsiloxane). However, many drugs have only limited or zero solubility within these hydrophobic liquids.

The use of so-called hydrophilic silicone oils, particularly those manufactured and distributed by Gelest, Inc can overcome this solubility issue if desired. As they have the same silicone backbone as the hydrophobic polydimethysiloxane silicone oil, these oils have the advantage of being very soluble within a carrier oil but are also able to dissolve hydrophilic compounds that might not otherwise be soluble in the polydimethysiloxane-based oil itself. Such compounds could be either small or very large biologicals encompassing a broad range of intended medical purposes. In this context, these compounds may dissolve in the oil of interest if a small amount of the hydrophilic oil is added to the overall silicone oil mixture. These drugs can exist as a solid that may benefit from limited dissolution and transport through the carrier hydrophobic liquid into the surrounding polymer wall of the capsule.

We have found that during the exposure of a dense polycaprolactone (PCL) capsule containing a hydrophobic silicone oil (Silikon 1000, purified polydimethylsiloxane, Alcon Laboratories, Fort Worth, TX) and Rose Bengal to phosphate buffered saline (PBS) over a period of 65 days no release is observed. Diffusion of the Rose Bengal out of the capsule does not occur because the Rose Bengal never dissolves in the oil. Thus, any silicone-oil insoluble drug contained within a purely hydrophobic environment will not be released by diffusion.

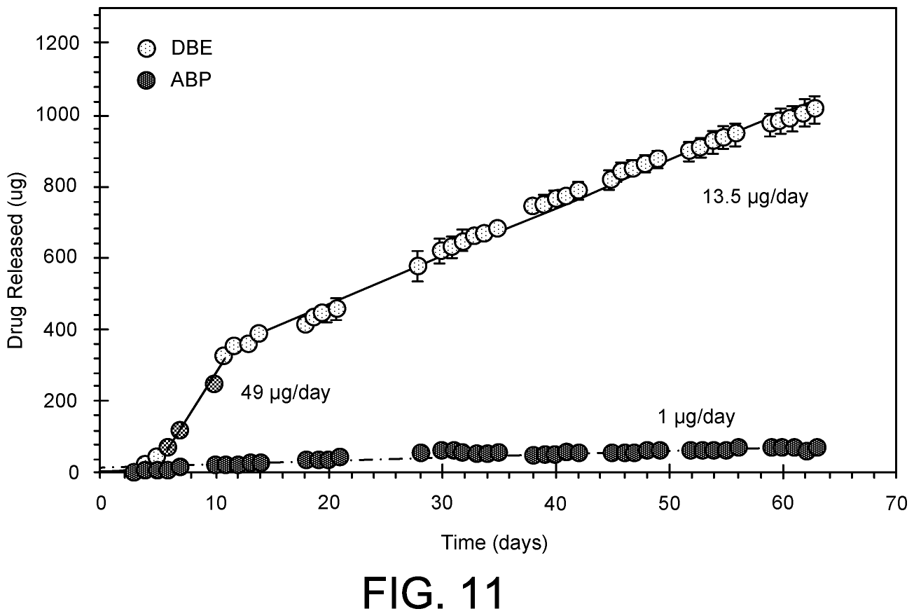

In contrast, FIG. 11 shows the net results of release from a dense polycaprolactone (PCL) capsule containing the hydrophilic silicone oil dimethylsiloxane-(60-70% ethylene oxide) block copolymer (DBE 712) and Rose Bengal into phosphate buffered saline (PBS) over a period of 64 days. Release is observed, indicating that diffusion of the Rose Bengal out of the solid occurs into the surrounding hydrophobic oil and then out of the capsule. Thus, a silicone-oil insoluble drug contained within this capsule can now be released by diffusion via this invention. In addition, the same experiment involving the hydrophilic silicone oil dodecylmethysiloxane-hydroxypolyalkyeneoxypropyl methylsiloxane copolymer (ABP-263) and Rose Bengal into phosphate buffered saline (PBS) over a period of 63 days showing that dissolution and release can also occur albeit at a slower rate.

This approach creates a solid ‘depot’ approach to drug release in which the capsule can continue to release the compound of interest through the wall via diffusion. The concentration of dissolved drug oil is then replenished by the remaining solid. This process can occur until the last of the solid form of the drug dissolves. If the desired rates of drug release are within the 10-30 μg/day range, the inclusion of a hydrophilic silicone oil can potentially allow for years of continuous release of a bioactive drug through a polymeric capsule wall.

There are multiple applications in drug release applications that require years of continuous, low-level release of highly biologically active compounds. Contraception in mammals is one such target. Chemotherapeutic release is another potential application in which the bioactivity of the compound must be preserved.

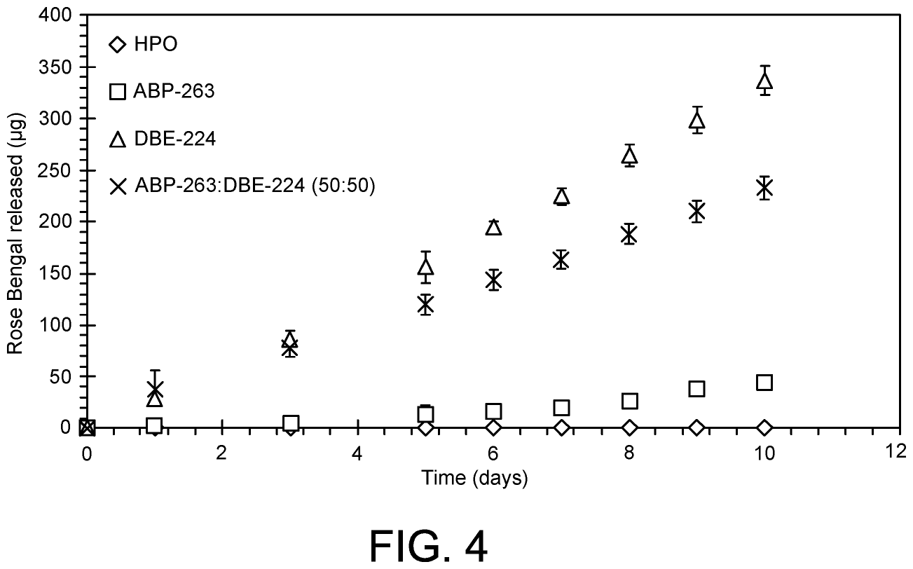

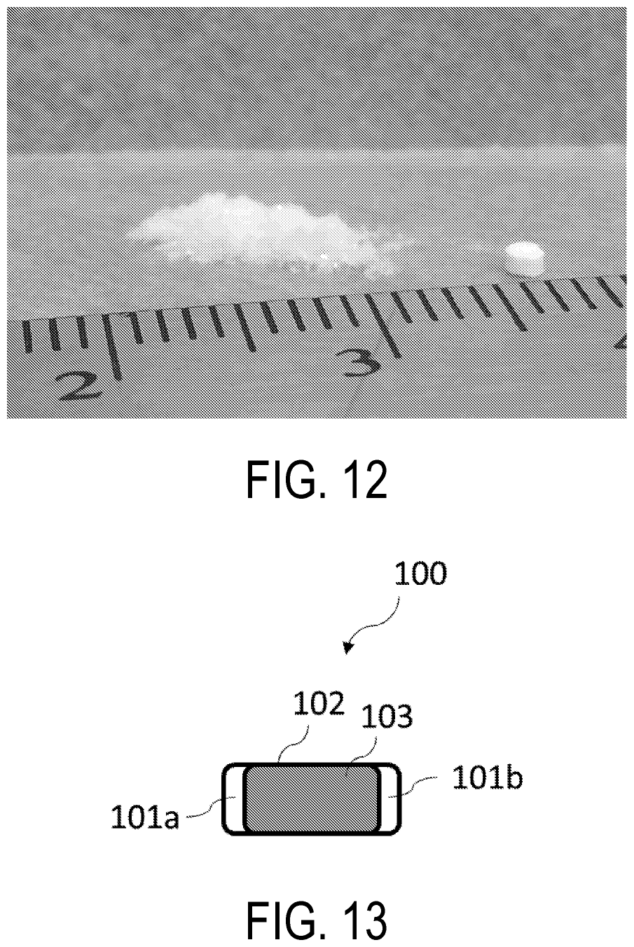

The use of powdered drugs can create problems to the inefficiency of powder packing. Having a high initial loading encourages high levels of saturation of the carrier fluid and thus drives greater diffusional release. Simple compaction of these powdered drugs as a means of minimizing drug volume while still retaining high levels of loading. FIG. 12 shows an image comparing the volume of 4 mg of loose deslorelin powder to the same weight of deslorelin following compaction in a small diameter metal die. Compaction allows for substantial reduction in volume as demonstrated allowing for higher capsule loadings without the space restrictions associated with incorporating loose, space consuming powder. In addition, the capsule in FIG. 4 was formed using 2 wt % silicone oil mixed in with the deslorelin. This both improves the hydrophobicity of the pellet (extending its resistance to drug degradation) while also lubricating the powder to achieve ˜100% density following compaction.

### Example 3: Nanoscale Porosity-Controlled Release from Sintered Electrospun Fibers

Engineered porosity in polymeric drug delivery vehicles can increase release rates above those of a solid matrix. 4-(2-hydroxyethyl)piperazine-1-ethanesulfonic acid sodium salt (HEPES) incorporation into blended polycaprolactone (PCL) and polyethylene terephthalate (PET) fibers generated controlled porosity and pore configuration within the wall of final sintered polymeric capsules.

Fibers of 88:10:2, 85:10:5, 82:10:8, 80:10:10 and 70:10:20 PCL:PET:HEPES ratios were electrospun and densified to create capsules; water treatment removed the embedded HEPES, resulting in nanoscale porosity.

Regardless of [HEPES], water absorption in capsules containing hydrophobic silicone oil was <1 wt % after a 120-day PBS exposure. In contrast, when two hydrophilic oils or their blends were used as carriers, water absorption ranked DBE-224>ABP-263:DBE-224 (50:50)>ABP-263, where DBE-224 is a dimethylsiloxane-(25-30/o ethylene oxide) block copolymer and ABP-263 is a dodecylmethylsiloxane-hydroxypolyalkyleneoxypropyl methylsiloxane copolymer. No release was observed when hydrophobic silicone oil was used as a carrier. Zero-order release was observed for all PCL:PET:HEPES ratios when DBE-224 was the carrier; as expected, net release was proportional to [HEPES]. COMSOL modeling suggested that diffusion is dominated by networks of discontinuous porosity versus interconnected porosity.

HEPES incorporation and subsequent removal provides a method to create controlled porosity in sintered electrospun fibers.

### Introduction

Polymeric delivery vehicles made via extrusion(1) or film formation(2) are commonly used to release drugs at specific rates. Depending on the effective size of the drug of interest, many of these vehicles do not attain desired release rates. The incorporation of porogens can sometimes eliminate this disadvantage (1,2). Techniques used include polymeric porogens (3,4), inorganic porogens (3-8) and freeze-drying techniques (9). Nanoscaled pores are often difficult to achieve at a useful overall scale when implementing porogens, because traditional techniques typically produce pores considerably larger than the nanoscale.

Inorganic salts have been used to incorporate nanoscale porosity; commonly used salts include gallium trichloride (7) or calcium carbonate (CaCO3) nanoparticles (10). Sodium chloride (NaCl) has been used to engineer porosity but has poor solubility in many of the solvents typically used in electrospinning (8,10). Soluble organic salts have been used to produce fiber bundles that improve neural cell growth (11) and can increase the interfacial strength and mechanical properties of polymer blends(12). Other uses of salt during electrospinning involve an increase in viscosity (13) and the reduction of fiber diameter by increasing solution conductivity (13,14).

To our knowledge, organic salts have not previously been used to engineer porosity for drug delivery in electrospun fibers. The inherent advantages of such salts to introduce controlled porosity via incorporation into the organic solvents used for electrospinning was utilized. This creates a means of controlling porosity in which the pores are initially confined to the location of the original electrospun fiber. The significance of this approach focuses on the general need for the slow release of specific drugs from implanted capsules. This release could be expected to continue for periods of months to years. Utilizing 4-(2-hydroxyethyl)piperazine-1-ethanesulfonic acid sodium salt (HEPES) as a pore-forming agent, we can direct and restrict the formation of pores in a post-sintered membrane as they follow the path of the original as-spun fibers. We utilize this technology to create designed porosity for drug release utilizing polycaprolactone (PCL):polyethylene terephthalate (PET):HEPES salt blends in the following ratios: 70:10:20, 80:10:10, 82:10:8, 85:10:5 and 88:10:2. The use of PCL:PET:HEPES blends allows the incorporation of a soluble salt to create small levels of porosity post-leaching and enhance drug delivery while retaining the desirable mechanical properties and lack of copolymerization imparted by the use of electrospun and sintered PCL:PET blends(15).

During these long periods of release, the preservation of drug bioactivity is targeted using relatively hydrophobic carriers within these capsules. In this work, we utilize blends of hydrophilic and hydrophobic silicone oils to achieve more precise control over the amount of moisture that a capsule can absorb which then influences internal dissolution and the net rate of drug release.

### Materials and Methods

### Scaffold Preparation

A total 5 wt % solids content solution consisting of PCL (Aldrich, St. Louis, MO, Mn=80,000), PET (Goodfellow, Coraopolis, PA, Intrinsic viscosity=0.84 dL g−1) and HEPES (Sigma-Aldrich, St. Louis, MO, MW=260.3 g mol−1) at a PCL:PET:HEPES ratio of 88:10:2 in 1,1,1,3,3,3-hexafluoro-2-propanol (HFP) (Oakwood Chemical, West Columbia, SC) was prepared by using continuous stirring overnight at 40° C. until completely homogeneous. This process was repeated for ratios of 85:10:5, 82:10:8, 80:10:10, and 70:10:20. All solutions were transferred to a 60 cm³ plastic syringe (BD Luer Lok, Franklin Lakes, NJ) tipped with a 20-gauge blunt needle (EFD, East Providence, RI). The following electrospinning conditions were then employed following the work of Chaparro et al.: a voltage potential of 24 kV, a flow rate of 5 mL h⁻¹ and a source to ground distance of 20 cm (16). Samples were spun at 500 rpm onto 6 cm long rotating 316 stainless steel rods. The diameter of the metal rod used for weight gain and dye release studies was 3.00 mm. In contrast, a rod having a diameter of 9.51 mm was used to produce samples for characterization of tensile properties. To remove any traces of residual solvents, a technique previously described was used (17). Briefly, all electrospun tubes were placed under vacuum (−30 in Hg) for 12 h at room temperature. The electrospun wall thickness was measured and determined to be 648±22 μm using a Keyence laser micrometer (Keyence, Model LS-7030) (18).

### Capsule Preparation

To obtain a dense polymer, as-spun tubes were sintered without removal from the metal rod. The sintering conditions followed our previous work (15) and were 100° C. for 3 h under −30 in Hg vacuum. The dense layer thickness was measured as described before (18). The obtained thickness was 109±9 μm. Once the polymer was fully densified on the circumference of the rod, a small amount of pressure applied to the sample surface tangential to the longitudinal direction of the rod was enough to loosen the metal-polymer interface. Following this, the samples were gently removed by deforming the tube to advance this air film along the length of the rod/deposition interface thus allowing the tube to slide off the rod. Tubes 2.50 cm in length were then cut from this initial tube length to create the capsules needed for the subsequent weight gain and dye release experiments.

To leach out the salt and create a porous sample, densified tubes containing 2, 5, 8, 10 and 20 wt % HEPES were exposed to distilled water at 37° C. in an incubator (Thermo Electron Corporation, Model 320) for 24 h while gently shaking in a vortex (VWR, Model VM-3000). The samples were then dabbed with a kimwipe then further dried overnight under vacuum and the percent salt removal calculated using the dried weights before and after water exposure.

To create closed capsules, both ends of the dense and porous polymer tubes were sealed following introduction of the drug payload of interest and/or an oil carrier. Sealing was achieved using a TTS-8 tabletop sealer (U Heat Seal Solutions, Corona, CA). The first seal was created using a heating time of 0.50 s and cooling time of 60 s. The model compound of interest and/or oil was added into the capsule and the final seal created using the prior heating and cooling parameters.

### Water Absorption

To study water uptake versus carrier fluid and time, various types of carriers were introduced into the capsules: polydimethylsiloxane, a hydrophobic oil (‘HPO’) with a viscosity of 1,000 cST (Clearco, Willow Grove, PA), and so-called ‘hydrophilic’ (‘HPI’) oils, either dodecylmethylsiloxane-hydroxypolyalkyleneoxypropyl methylsiloxane copolymer with a viscosity of 1,000-4,000 cST (‘ABP-263’) (Gelest, Morrisville, PA) or dimethylsiloxane-(25-30% ethylene oxide) block copolymer with a viscosity of 400 cST (‘DBE-224’) (Gelest, Morrisville, PA). Blends of DBE-224:ABP-263 in a 50:50 volume ratio were also created. A micropipette was used to introduce a total volume of 40 μL of each oil or their blends into the PCL:PET:HEPES capsules (n=3). The weight of each sample was recorded at 0.10 mg precision prior to media exposure in 15 mL glass vials. The media consisted of 10 mL 1× Phosphate Buffered Saline (PBS, pH ˜7.4) (Fisher, Fairlawn, NJ) with 0.02 wt % sodium azide (Sigma-Aldrich, St. Louis, MO) added to minimize bacterial growth. Each capsule was removed from the glass vials, carefully dabbed with a kimwipe and weighed following specific time exposures (1, 3, 7, 14, 21, 28, 35, 49 and 120 days) at 37° C. in an incubator (Thermo Electron Corporation, Model 320). They were then re-exposed to the same conditions until the subsequent sampling point.

### Tensile Testing

Tensile specimens originally spun on 9.51 mm diameter metal rods were obtained from 80:10:10 PCL:PET:HEPES tubes from as-spun and fully densified samples. The polymer tubes were removed from the rod by cutting along the longitudinal direction using a #15 scalpel. After gently flattening the resulting sheet, a tensile dog bone-shaped punch 12 mm in gauge length and 3.0 mm in width was used to create tensile samples (n=6). Sample thickness was measured using a digital micrometer by placing the gauge length of the tensile specimen between two glass microscope slides of known thickness. Tensile tests were performed in a load frame (Instron, model 1000R12) using a 250 lb load cell (Test Resources, MTestW R system) following the ASTM D882-10 standard. Samples were secured using a lightweight carbon fiber grips (A2-166 Fiber Clamp Assembly, Instron) and the test executed at a loading rate of 5.95 mm min−1 while recording every 0.0500 seconds. Elongation to failure, ultimate tensile strength (UTS) and modulus were acquired in this manner. As a control sample, as-spun and densified 88.89:11.11 PCL:PET capsules were fabricated as described previously and underwent the same mechanical testing(15).

### Model Compound Release

To study in vitro release behavior from these capsules, Rose Bengal sodium salt (RB) (95%, Aldrich, mw 1017.6 g mol−1, St. Louis, MO) was used as a model of a higher molecular weight drug. A custom-made punch and die (PARR Instrument Company, Moline, IL) with a diameter of 1.5 mm and a length of 6.35 mm was used to make pressed pellets of RB (15.57±0.63 mg). A load frame (Instron, model 1000R12) with a 250 lb load cell (Test Resources, MTestW R system) at a rate of 0.46 mm min−1 and a 20 s hold at 5.0 lb was used to compress them. The RB pellets were then placed inside the water-treated PCL:PET:HEPES capsules (n=3) along with 40 μL of the oil of interest. They were then sealed, added to 15 mL glass vials containing the same media and incubated at 37° C. as before. At specific time intervals, aliquots of 100 μL were extracted from the vial solution, stored in a 96-well plate (Costar®, Salt Lake City, UT) and the media completely exchanged with fresh solution. The amount of RB released versus time was measured using a plate reader (SpectraMax®, M Series, Molecular Devices) by determining the fluorescence at excitation and emission wavelengths of 540 and 575 nm, respectively. Densified 88.89:11.11 PCL:PET capsules were also tested in the same manner in our previous work to serve as a control group(15). A calibration curve was established for RB with concentrations in the range of 1×10⁻⁵ to 0.1 μg mL⁻¹. If the RB concentration was above 0.1 μg mL⁻¹, the solution was diluted to be inside the calibration curve range, measured and corrected by the dilution factor. Experimental results were considered to have zero-order release kinetics if the obtained cumulative release of the model drug over time had a linear R2>0.9.(19)

COMSOL (v5.3a) was used to simulate RB release from capsules with varied initial PCL:PET:HEPES ratios (88:10:2, 85:10:5, 82:10:8, 80:10:10 and 70:10:20) using DBE-224 as the carrier oil. Simulations were performed using a capsule cross-section with an inner diameter of 3 mm and a capsule shell thickness of 109 μm. Release occurred into a PBS reservoir(20) with a 19 mm diameter (representing the inner diameter of the glass vial). The [RB] in the outer PBS reservoir was monitored versus time and was converted into the corresponding weight released by the capsule. RB diffusivity in PBS was assumed to be 4.8×10⁻¹⁰ m² s⁻¹(20)

The observed water uptake versus time was assumed to be directly proportional to the amount of RB available for diffusion from the capsule interior. The water uptake (in grams) versus time (in seconds) for the first 10 days was fit with a second-order polynomial containing a proportionality constant fixed at k=2,000 for all simulations.

[RB]=k(−2.8578×10−14t2+3.3816×10−8t+3.3717×10−3) (1)

In this context, the solubility of RB in DBE-224 was assumed to be negligible compared to the solubility of RB in PBS. Since the weight gain was statistically insignificant for the three capsule compositions studied (PCL:PET:HEPES of 80:10:10, 85:10:5 and 88:10:2), it was assumed that all modeled capsule compositions would experience identical PBS uptake. Therefore, the data for the 85:10:5 capsule was used to represent all five capsule compositions.

Release simulations were first performed as if the capsules had interconnected porosity. In this case, the volume percent of porosity was estimated using the density of each component. Since the density of HEPES was not given, it was assumed to be similar to that of sodium persulfate (˜2.6 g cm³). In these cases, the main pathway for diffusion was assumed to be through PBS-filled interconnected porosity with an effective diffusivity related to the porosity by the Millington-Quirk model. Additional release simulations were performed using modeled capsules that did not exhibit interconnected porosity. In these cases, the effective diffusivity of RB through the capsule wall was assumed to vary according to [HEPES].

### Scanning Electron Microscope (SEM) Analysis

A cross-section image of each capsule wall was obtained by placing the specimen in liquid nitrogen for 5 minutes followed by fracture. Samples were placed on conductive carbon tape (SPI Supplies, West Chester, PA) adhered to aluminum SEM sample mounts (TED Pella, Redding, CA) and sputter coated twice (EMS, model 150TS) with gold-palladium (60:40) for 30 seconds at an emission current of 30 mA. Microstructure analysis was observed under an SEM microscope (FEI Nova 400) at an accelerating voltage of 5 kV. Fiber diameter (n=50) and pore size (n=200) was measured via ImageJ (version 1.6.0) with respect to the scale bar at a magnification of 30,000×.

### Statistical Analysis

The presence or absence of statistical difference between water absorption behavior was performed by means of one-way analysis of variance (ANOVA) followed by Tukey-Kramer HSD tests. This analysis was performed with JMP Pro (v. 14.0.0). The effect of oil carrier composition was assessed for each specific PCL:PET:HEPES composition, while the effect of capsule composition was assessed among capsules with the same oil carrier.

The measured mechanical properties (ultimate tensile strength, elongation to break and modulus) were analyzed with R statistical software (v. 3.3.2) (21) using a one factor linear model for the 6 different capsule conditions. To achieve error's normality and homoscedasticity, elongation to break was transformed with an inverse function, while the ultimate tensile strength and modulus data were logarithmically transformed. For multiple comparisons, a Tukey adjustment was applied.

In each case, significance was attained at a value of p<0.05. All data is presented as mean±standard deviation.

### Results

### Microstructure Analysis

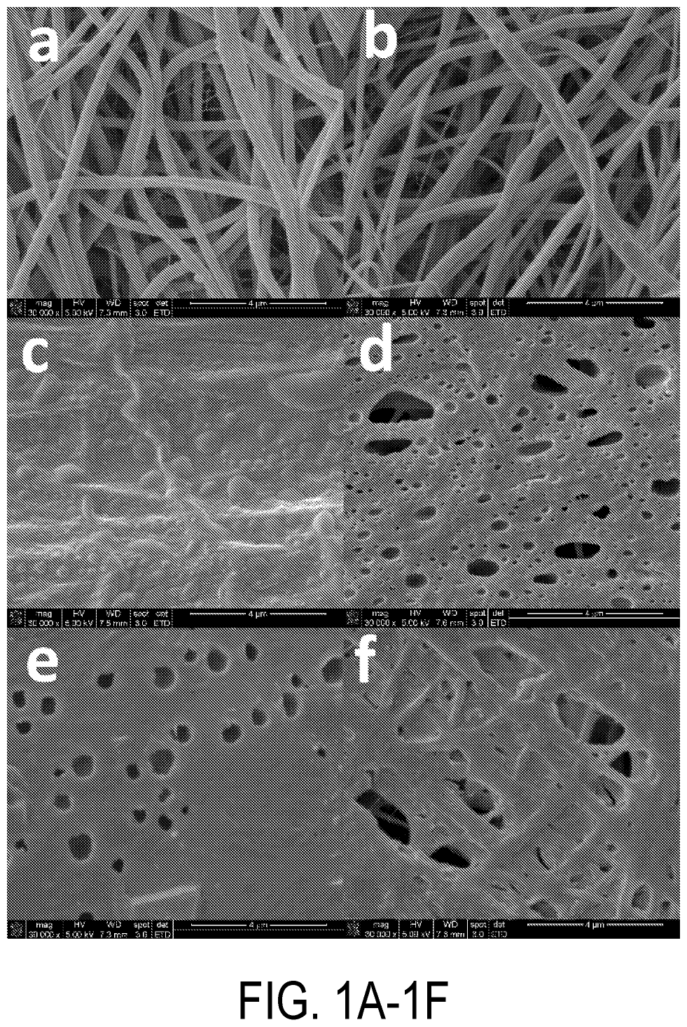

HEPES salt was readily soluble in HFP and this allowed it to be easily incorporated into the PCL:PET blend solutions at various PCL:PET:HEPES ratios. FIG. 1 shows the microstructure of PCL:PET:HEPES (80:10:10) for as-spun and fully densified samples pre- and post-water treatment. The as-electrospun fibers (FIG. 1a) appear slightly rough due to the salt content and have a diameter of 273±96 nm. Occasional fiber bundling is observed for the as-spun fibers (FIGS. 1a-b). After heat treatment under vacuum, the PCL:PET:HEPES fiber layer becomes fully densified (FIG. 1c). Salt agglomerates are visible in the microstructure as small embedded spheres. After the as-spun and fully dense samples (FIGS. 1b and 1d) are exposed to water, the observed weight loss is largely in accordance with the initial [HEPES](Table 1) and pores are created having average diameters of 134±33 and 252±256 nm, respectively. Some pore coalescence into micro-scale pores during sintering was also evident with sintered, water-treated samples having a wide range of pore sizes ranging from ˜100 nm to greater than 2 μm. The average pore size for densified, water-treated samples having varied initial [HEPES] is 435±114 nm and 2117±312 nm for the 88:10:2 and 70:10:20 samples, respectively. Close examination of the images reveals that the surprisingly larger apparent pore size for the 88:10:2 sample is likely a result of the highly variable pore morphology; the 88:10:2 sample contains primarily round, isolated pores in contrast to the more angular pores observed for higher HEPES contents (FIGS. 1d and 1f) that appear to extend well beneath the surface.

TABLE 1
Salt removal of heat treated PCL:PET:HEPES blends
after water exposure at 37° C. for 24 hours.

| PCL:PET:HEPES | Weight loss (%) |
|---|---|
| 88:10:2 | 1.66 ± 0.26 |
| 85:10:5 | 4.83 ± 0.30 |
| 82:10:8 | 7.67 ± 0.33 |
| 80:10:10 | 9.93 ± 0.63 |
| 70:10:20 | 25.24 ± 1.72 |

### Water Absorption

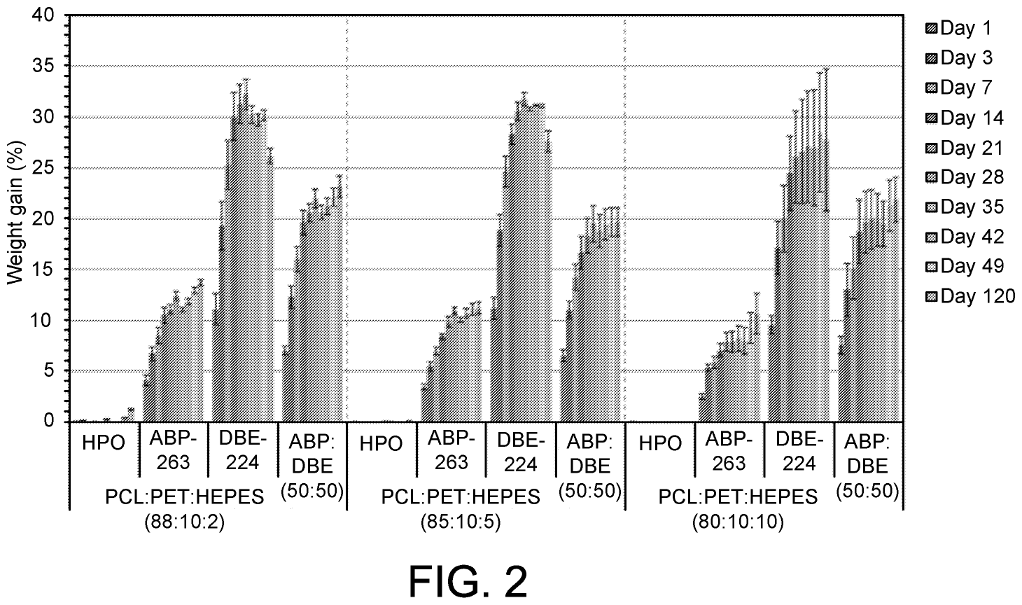

The water absorption behavior of the PCL:PET:HEPES capsules containing engineered porosity is shown in FIG. 2. For each capsule composition, the oil carrier had a significant effect on water absorption (one-way ANOVA, p<0.05). Negligible water absorption by HPO was observed up to day 49 regardless of HEPES loading; weight gain remained <1% after 120 days. Independent of the initial HEPES percentage, all modified oils show statistically greater (Tukey-Kramer HSD, p<0.05) water absorption than the HPO carrier versus time. In terms of the HPI carriers, the weight gain ranking was DBE-224>50:50 blend>ABP-263 with statistically significant differences (Tukey-Kramer HSD, p<0.05) among oil carriers at each time point for 88:10:2 and 85:10:5 capsules regardless of initial [HEPES]. Surprisingly, the initial amount of HEPES in capsules containing either DBE-224 or the 50:50 blends does not have a statistically significant effect on water absorption (one-way ANOVA).

### Mechanical Properties

Table 2 shows the mechanical properties of 80:10:10 PCL:PET:HEPES under various conditions (as-spun versus sintered and water treated versus non-water treated) as compared to the 88.89:11.11 PCL:PET control. All UTS results are statistically different with the exception of water treated and non-water treated as-spun PCL:PET:HEPES. Moreover, although the sintered PCL:PET control does not have a statistically different modulus compared to water treated sintered PCL:PET HEPES, all other moduli are statistically different. In regards to elongation, sintered PCL:PET has a higher elongation (p<0.05) than all other conditions, but the remaining samples are not statistically different from one another.

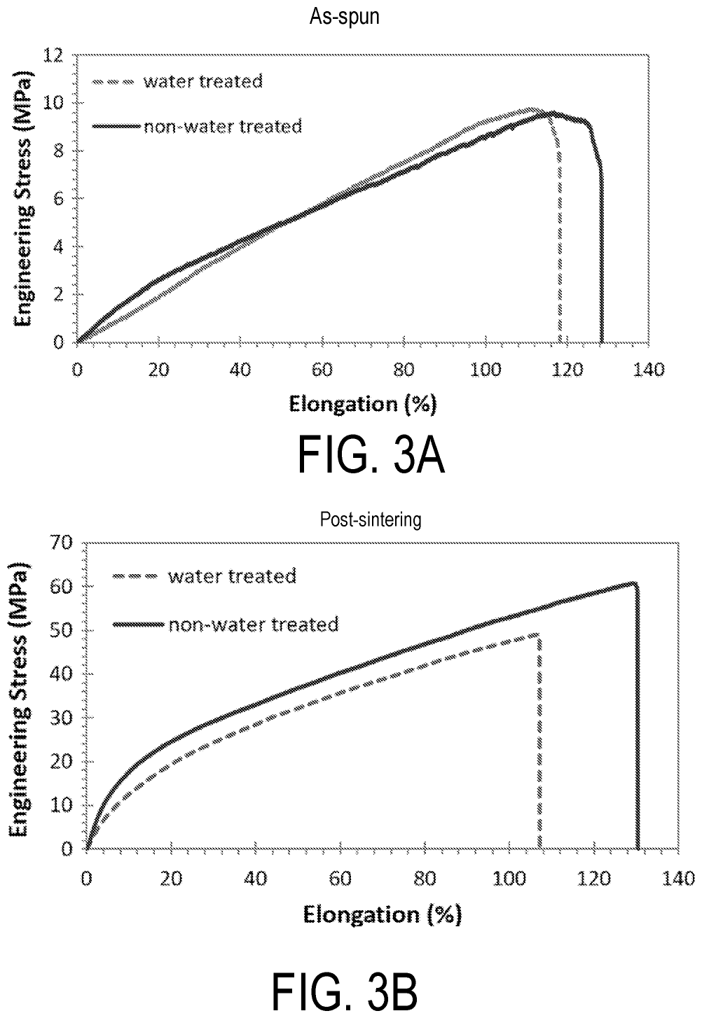

Sintering caused the expected improvements in mechanical properties for 80:10:10 PCL:PET:HEPES pre- and post-salt leaching. Additionally, the presence of HEPES generally leads to increased UTS and modulus. Representative curves of failure behavior are shown in FIG. 3. Upon reaching maximum elongation, all samples exhibited brittle failure although this was more pronounced in the fully dense samples.

TABLE 2
Mechanical properties of various 80:10:10 PCL:PET:HEPES
and 88.89:11.11 PCL:PET conditions.

| | | UTS | Elongation | Modulus |
|---|---|---|---|---|
| | Condition | (MPa) | (%) | (MPa) |
| PCL:PET:HEPES | | | | |
| (80:10:10) | | | | |
| As-spun | Water treated | 9.28 ± 0.95 | 117 ± 15 | 11.65 ± 1.56 |
| | Non-water | 9.69 ± 1.22 | 111 ± 11 | 19.15 ± 4.41 |
| | treated | | | |
| Fully | Water treated | 47.19 ± 4.32 | 108 ± 4 | 141.90 ± 22.02 |
| densified | Non-water | 60.44 ± 4.05 | 114 ± 10 | 314.45 ± 19.81 |
| | treated | | | |
| PCL:PET (88.89:11.11) | | | | |
| (15) | | | | |
| As-spun | — | 2.59 ± 0.22 | 94 ± 8 | 2.58 ± 0.44 |
| Fully densified | — | 28.01 ± 1.99 | 154 ± 33 | 177.46 ± 20.15 |

### In Vitro Release

The in vitro RB release of 80:10:10 PCL:PET:HEPES post-leaching using HPO, ABP-263, DBE-224 and a blend of these two HPI oils in a 50:50 volume ratio is shown in FIG. 4. A release of 0.00 μg day⁻¹ is observed when HPO is used as a carrier. Meanwhile, release rankings when HPI oils are used is DBE-224>50:50 blend>ABP-263 at release rates of 33.81, 22.49 and 4.26 μg day⁻¹, respectively. A linear correlation is observed for capsules containing DBE-224 at an R²>0.995 (Table 3) showing that they exhibit zero-order release.

TABLE 3
In vitro release rates from PCL:PET:HEPES (80:10:10) capsules
post-salt leaching using different oil-based carriers.

| Carrier | Release rate (μg day⁻¹) | R² |
|---|---|---|
| HPO | 0.00 | — |
| ABP-263 | 4.26 | 0.9122 |
| DBE-224 | 33.81 | 0.9981 |
| ABP-263:DBE-224 (50:50) | 22.49 | 0.9972 |

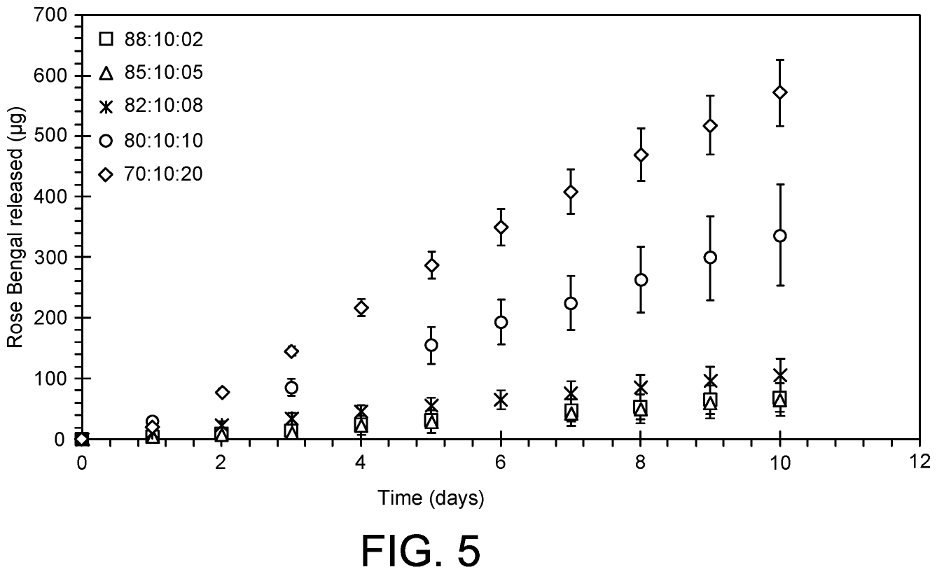

RB release from different HEPES ratios after salt leaching using DBE-224 as the carrier is shown in FIG. 5. Capsules made using 2 and 5% HEPES exhibit similar release post-leaching at rates of 6.87 and 7.30 μg day⁻¹, respectively. Capsules made using 8, 10 and 20% HEPES show release rates of 10.56, 33.81 and 70.73 μg day⁻¹, respectively. A zero-order release is also attained for these capsules following salt removal (R²>0.99, Table 4).

TABLE 4
In vitro release of RB from PCL:PET:HEPES
capsules containing DBE-224 carrier oil.

| PCL:PET:HEPES | Release rate (μg day⁻¹) | R² |
|---|---|---|
| 88:10:2 | 6.87 | 0.9938 |
| 85:10:5 | 7.30 | 0.9911 |
| 82:10:8 | 10.56 | 0.9993 |
| 80:10:10 | 33.81 | 0.9981 |
| 70:10:20 | 60.73 | 0.9956 |

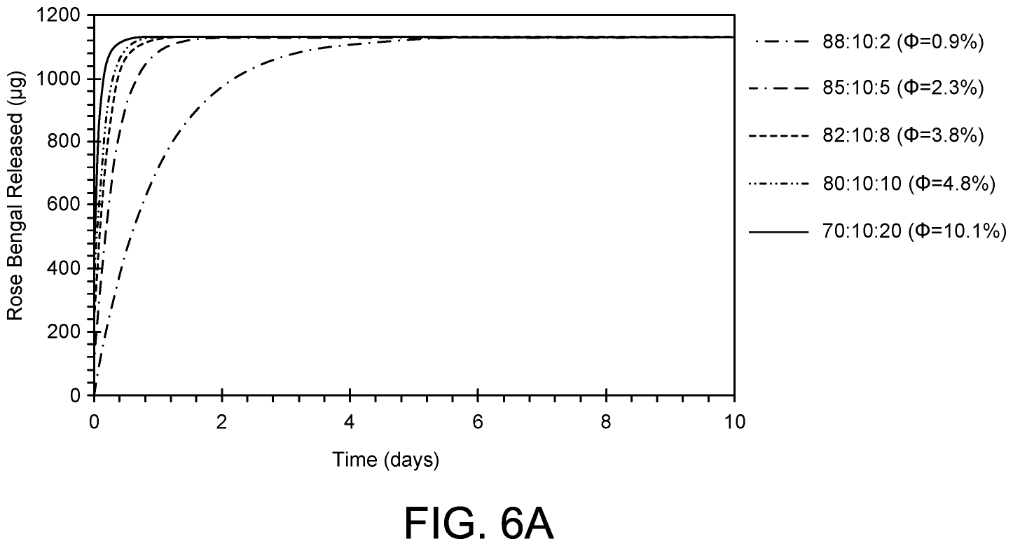

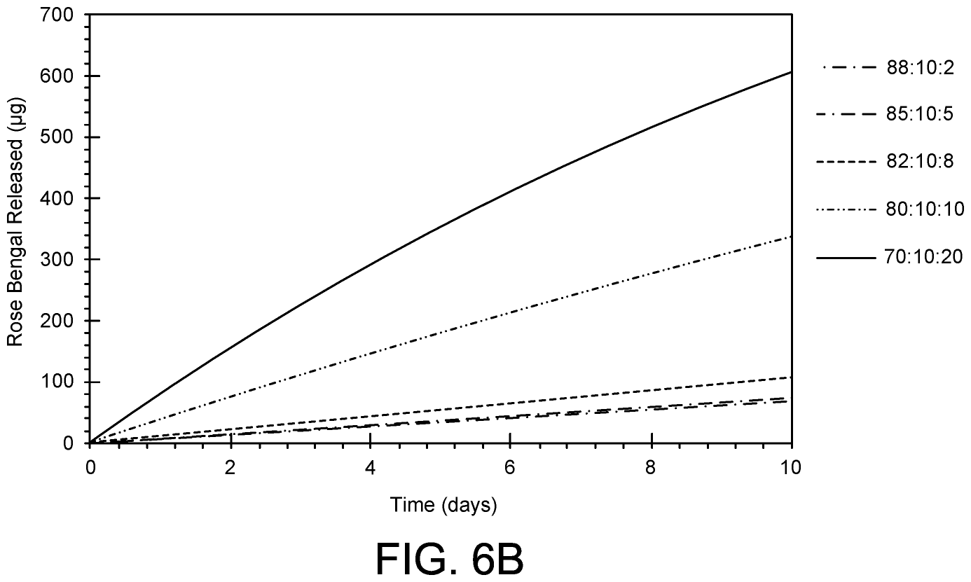

FIG. 6a shows the simulated RB release for the five capsule compositions assuming interconnected porosity. These profiles display burst release which corresponds poorly to the experimentally observed zero-order release.

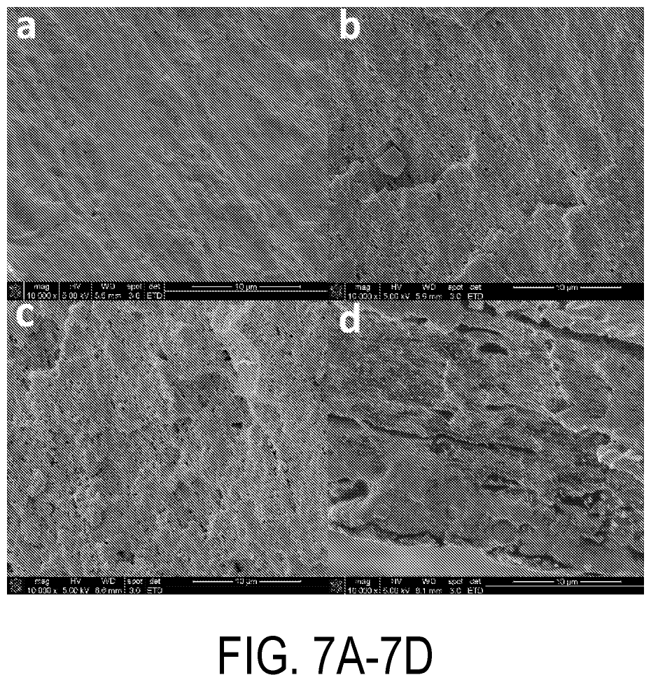

In contrast, FIG. 6b shows the simulated release assuming a lack of through-thickness interconnected porosity in the capsule wall. In this case, the effective diffusivity was assumed to vary directly with [HEPES]; the effective diffusivity was varied to establish those values that resulted in the observed total RB release after 10 days. The effective diffusivity values were found to be similar for 2 and 5 wt % HEPES (5.55×10⁻¹⁵ and 5.95×10⁻¹⁵ m² s⁻¹, respectively). The effective diffusivity value was 1.6 times higher for 8 wt % HEPES than for 2 wt % HEPES. The effective diffusivity values increased substantially for 10 and 20 wt % HEPES, yielding values ˜6- and ˜12-fold (respectively) higher than those of 2 wt % HEPES. Note that the absolute values of the effective diffusivities rely on the chosen proportionality constant. However, these effective diffusivity values can be compared and the shape of the release profile assessed. In general, in the absence of interconnected porosity, these simulated profiles yield the same zero-order release observed experimentally. FIG. 7 shows the cross-sections of the 5, 10 and 20% HEPES blends after leaching. As the salt content increases, the porosity and degree of interconnectivity become more evident. The capsule initially containing only 5% salt exhibits relatively little porosity, while its 10 and 20% counterparts show evidence of more extensive porosity. FIG. 7d shows the cross-section of the capsule containing 20% HEPES prior to salt leaching. The black areas represent the presence of salt found throughout the microstructure.

### Discussion

Porogen leaching is widely used to create porous polymeric scaffolds(3,22-25). Yin et al. created porous scaffolds by incorporating NaCl particles into poly(ethylene glycol) (PEG) and poly(lactic acid) (PLA) prior to extrusion (3). Leached via water exposure to remove the salt, this created porous scaffolds having pores in the range of 8-10 μm in diameter. While the pore sizes were the same regardless of the salt loading, the connectivity of the different amounts of NaCl (1.25 to 5.00 wt %) in the extruded cylinders was greater than 97% due to the uniform salt distribution throughout the sample. However, the main hindrance of using extrusion—even to incorporate salts into the matrix—is the likelihood of chemical alterations to both thermosensitive polymers and their drug payloads (26-28).

The use of NaCl as a porogen often suffers from the wide variability in the resulting pore size post-leaching (6,8). In addition, due to the poor solubility of NaCl in solvents commonly used for electrospinning PCL (29), pore size post-leaching can only be influenced by the initial salt particle size (6). Wang et al. showed that salt incorporation into large (>2 μm) diameter PCL electrospun fibers was difficult and possible only for salt particles <1 μm in diameter (10). No salt retention was observed by small diameter (<0.7 μm) PCL fibers. Nam et al. demonstrated that the mechanical introduction of NaCl crystals during the electrospinning of PCL fibers resulted in delamination immediately following water treatment; sintering at 45° C. for 10 minutes prior to salt removal prevented this delamination (8). Physical incorporation of NaCl into our system would increase drug release; however, the variable pore sizes demonstrated in prior studies (6,8,10) suggest zero-order release would be unlikely. Seo et al. demonstrated such behavior when different amounts of water-soluble PEG were incorporated into polyurethane (PU) films to produce pore sizes up to 14 μm. A smaller pore size around 1.5 μm was observed when 10 wt % PEG in PU was used; elution of paclitaxel showed a biphasic behavior consisting of a burst release during the first few hours followed by a more gradual release out to 19 days (25).

Low concentrations of inorganic or organic salts in electrospinning solutions are often used to control conductivity, viscosity, fiber diameter and ‘spinnability’(11,14,30). The presence of the soluble organic salts ethylamine hydrochloride and tetraethylammoniumbromide created bundled PCL fibers (11) and improved the spinnability of PU (30), respectively. Inorganic CaCO3 nanoparticles (≤100 nm)(10) and the soluble inorganic gallium trichloride (7) salt created small internal porosity in polymer fibers post-leaching.

HEPES is widely used as a biocompatible buffering agent due to its ability to maintain pH in the critical region between 7.4-7.8 (31). Interestingly, HEPES-based pore formation has occurred in polymers by simply exposing them to buffers containing HEPES salt (9,32); the deliberate use of this salt as a porogen during polymer processing has not been reported. In this study, we found that HEPES salt could be dissolved in the common electrospinning solvent, HFP. Desired ratios of the ternary PCL:PET:HEPES could thus be easily prepared.

### Microstructure Analysis

The ternary system consisting of 80:10:10 PCL:PET:HEPES demonstrated the desired ‘spinnability’ and the anticipated decrease in fiber diameter compared to the 88.89.11.11 PCL:PET control; an average 2-fold decrease in diameter was noted (15). Similar behavior was reported by Liu et al. in which fiber diameter decreased after the introduction of ampicillin sodium salt (34). FIG. 1a shows the uniformly distributed salts in the as-spun fibers of this ternary system resulting from the high solubility of HEPES in HFP. Cho et al. saw similar uniform salt distribution when using ethylamine hydrochloride as the soluble salt to create salt-induced electrospun patterned bundles (11). Chou et al. observed a rough fiber morphology similar to our as-spun fibers when salt was present on the surface (11). Once water treated, the as-spun samples also display a well-distributed nanoscale porosity along the entire fiber surface implying that the ternary system was well dispersed in solution before spinning.

Once sintered under vacuum, two distinctive morphological features are present: residual fibers and partial HEPES salt agglomeration. When PCL:PET was heat treated, the PCL melted and eliminated porosity, likely leaving behind residual PET-rich fibers. Note that we have previously revealed evidence of residual fiber morphology in sintered PCL:PET compositions following selective solvent exposures (15). Salt agglomeration appeared to occur in two distinct forms (i) spheres resulting from apparent salt agglomeration and (ii) nanoscale pores running along the path of the initial as-spun fiber structure (FIG. 1). These two types of agglomeration became more obvious when the heat-treated samples were exposed to water. Two separate studies by Yin et al. and Barbanti et al. on extruded samples containing NaCl demonstrated that even when the sample was initially well mixed (melt mixed and heated during extrusion, respectively), salt agglomeration occurred creating pores larger than the initial particle size (3,24). Although we do observe some agglomeration, the majority of HEPES salt incorporated into the polymer blend solution resulted in <1 μm diameter porosity after leaching. Wang et al. observed that electrospinning PCL+CaCO₃ nanoparticles resulted in nanoparticles embedded primarily within the surface of the fibers. When these nanoparticles were removed, small pores were left behind in a linear pathway following the fiber direction (10). Post-sintering, this porosity sometimes appears to follow the original direction of our as-spun fibers, which may be a result of residual fiber morphology. However, in other areas, salt diffusion and aggregation appeared to occur during sintering, randomizing the direction of salt aggregates (FIG. 1d).

Consistent with previous reports, in our compositions leaching by water exposure removes the majority of the salt (3,22). In the case of compositions initially containing 10 wt % HEPES or less, traces of salt appeared to be left within the matrix; similar results have been observed for other salt porogens at low concentrations (3). In contrast, the 70:10:20 specimens demonstrated >20% weight loss after removal of the HEPES salt. Similar behavior was observed by Peng et al. who proposed that higher NaCl loadings in their PLA:PVA system caused salt to surround some of the polymer and remove it during leaching, producing pores larger than the initial salt particle size (5).

### Water Absorption

The water absorption of various oil blends containing hydrophobic silicone oil and the so-called hydrophilic silicone oils (ABP-263 and DBE-224) encapsulated into fully densified PCL:PET:HEPES capsules at ratios of 82:10:2, 85:10:5 and 80:10:10 is shown in FIG. 2. When HPO is the carrier, water absorption remains ˜zero at 49 days independent of the salt concentration prior to leaching. Similar behavior was observed for dense PCL capsules (33); the presence of HEPES-induced porosity throughout the matrix does little to enable water-based weight gain when HPO is the carrier fluid. In contrast, the use of HPI oils as carrier fluids in capsules containing HEPES-produced porosity allowed substantial water uptake. The presence of a more hydrophilic carrier fluid apparently enables substantially greater water infusion in the presence of engineered porosity. Similarly, Valdes-Garcia et al. demonstrated that introduction of a hydrophilic compound (almond skin residues) into PCL films increased water absorption (35).

### Mechanical Properties

The mechanical properties of as-spun 80:10:10 PCL:PET:HEPES, either water or non-water treated, demonstrated improved UTS and modulus versus the non-HEPES control group having the same PCL:PET content (88.89:11.11) from our previous work (15). In contrast, elongation was not statistically different. The UTS of the 80:10:10 showed a 4-fold increase in either the water or non-water treated forms; the modulus increased 5- and 7-fold for the water and non-water treated forms, respectively. For the non-water treated samples, the UTS and modulus increases are likely due to the presence of trace HEPES sodium salt that could increase secondary bonding between polymer chains. Interestingly, the water treated 80:10:10 PCL:PET:HEPES samples exhibited a higher UTS and modulus than the 88.89:11.11 PCL:PET control despite their extensive internal porosity. Table 1 suggests that small amounts of HEPES sodium salt could remain following water treatment. Also, the heavier (238.30 g mol−1) anionic portion of the HEPES sodium salt could have been preferentially leached out during water treatment while allowing much smaller cationic sodium ions to remain. These sodium ions could interact with the polymer chains—particularly with the ester groups of PCL and PET—to increase secondary bonding and result in the enhanced mechanical properties. Alternatively, the aforementioned 2-fold fiber diameter decrease observed following HEPES incorporation could generate greater frictional interactions that increase the modulus and UTS of the 80:10:10 compositions (36).

Similar behavior is observed for heat treated PCL:PET:HEPES with and without salt removal. When compared to heat treated PCL:PET at a 88.89:11.11 ratio, the 80:10:10 non-water treated specimens displayed an increase of 2.2- and 1.8-fold for UTS and modulus, respectively. In accordance with these results, Cazan et al. demonstrated that the tensile strength, impact strength and compression are improved when sodium dodecyl sulphate salt is incorporated into PET blended with rubber and polyethylene (12). Meanwhile, the water treated samples show a 1.7-fold increase for UTS when compared with PCL:PET (88.89:11.11) (15); this increase is likely a result of the smaller fiber diameter and increased secondary bonding from residual salt species still present in the water treated samples. However, the difference in modulus between these two groups is similar, this is likely a result of several competing factors. The incorporation of small pores would be expected to decrease the modulus, but the smaller fiber diameter and the presence of residual salt species could both be expected to increase the modulus. Interestingly, the mechanical behavior of the PCL:PET and the PCL:PET:HEPES samples is very similar (FIG. 3) (15).

### In Vitro Release

The in vitro release behavior resulting from encapsulation of HPI, HPO and their blends from the 80:10:10 PCL:PET:HEPES ratio post salt-leaching was studied (FIG. 4). Release of RB from the HPO carrier could not be measured. Even though the introduction of pores typically increases drug release (2,23,38), the hydrophobic oil apparently eliminated water infusion and, therefore, release became negligible. In contrast, when HPI oils and their blends are present, zero-order RB release was achieved. Due to the increased ability of these oils to absorb water, the dissolution and release of RB was enhanced. Therefore, the ranking of RB release from the PCL:PET:HEPES (80:10:10) after salt removal correlates to water absorption (FIG. 2) and was DBE-224>ABP-263:DBE-224 (50:50)>ABP-263. These results suggest that use of an appropriate oil or oil blend can be used to achieve and control zero-order release. Theoretically, if we assume that zero-order release from these capsules (regardless of material degradation) can be sustained until the payload (15.57 mg) is consumed, release could continue for 10.42, 1.97 and 1.31 years for capsules containing ABP-263, ABP-263:DBE-224 (50:50) and DBE-224, respectively.

the porosity of PCL capsules through was previously controlled using varied sintering temperatures and observed the resulting effect on drug release. Additionally, it was demonstrated that the molecular weight and physicochemical properties of three different model compounds greatly affected release behavior (16). In this work, varied HEPES salt ratios achieved the introduction of varied porosity into PCL:PET blended capsules, and release of a single model compound was studied. Due to its ability to obtain a zero-order release profile and a high release rate, the HPI oil DBE-224 was chosen as a carrier for all subsequent experiments. FIG. 5 demonstrates that regardless of the amount of HEPES used prior to leaching, zero-order release is obtained. Interestingly, samples with 2 and 5% HEPES exhibit essentially the same release rate, but the release rate increases in a non-linear fashion as [HEPES] increases from 5 to 20%. The SEM shows some pore connectivity for higher HEPES concentrations (FIG. 7b-c), but it is unclear whether any true through-thickness interconnectivity is present.

COMSOL simulations further explored the role of [HEPES] in controlling release rate. If true interconnected porosity was present in any of the ternary compositions, an initial burst release would be expected (FIG. 6a). Since zero-order release was instead observed, it appears that true through-thickness interconnected porosity was likely absent for even the 80:10:10 and 70:10:20 PCL:PET:HEPES capsules. This observation is supported by statistically similar water uptake versus [HEPES](FIG. 2). Additionally, simulations of capsules without interconnected porosity (FIG. 6b) exhibit zero-order release as observed experimentally (FIG. 5). For these simulations, there was only a slight increase in effective diffusivity from 2 to 5 wt % HEPES. However, the effective diffusivity increased dramatically with [HEPES] from 8 to 20 wt %. Therefore, the effective diffusivity increased with initial salt concentration in a non-linear fashion.

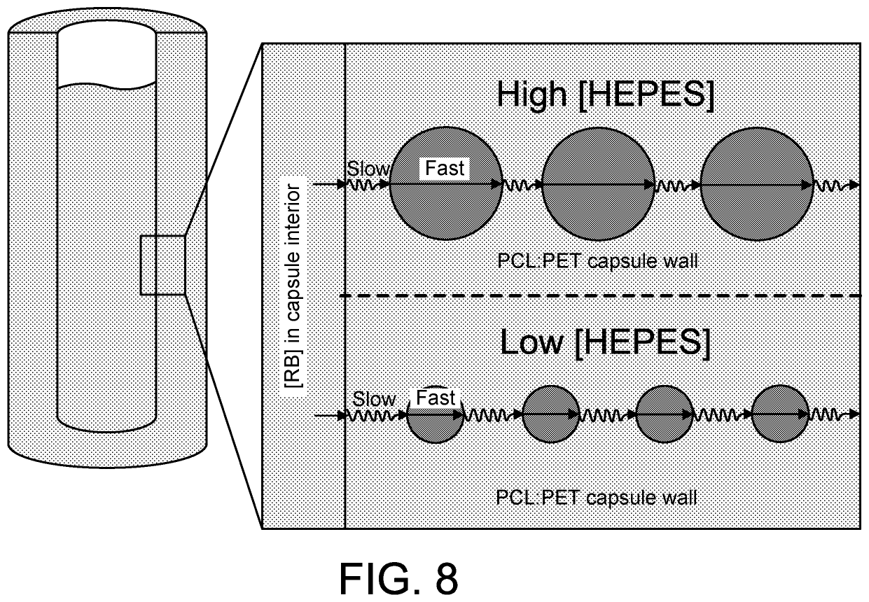

However, some degree of pore coalescence was observed in the 80:10:10 and 70:10:20 capsule cross-sections (FIG. 7). Therefore, even though true through-thickness interconnectivity does not appear to exist, small regions of pore interconnectivity are possible. These small regions of interconnectivity can hydrate, and RB diffusion through the hydrated pores would be orders of magnitude faster than through solid PCL:PET. Therefore, we hypothesize that a network of hydrated pores surrounded by PCL:PET exists. Diffusion then occurs in two forms: (i) rapid motion through hydrated pores followed by (ii) significantly slower rate-limiting diffusion through PCL:PET until the next hydrated pore is reached. This motion can be described as saltatory, a term normally used to describe the node-to-node travel of action potentials along nerve fibers (39). As [HEPES] increases, the size of the hydrated regions increases while the distance between them decreases, allowing for progressively faster release rates. A schematic detailing the proposed saltatory motion is shown in FIG. 8. Proposed saltatory motion of RB through a polymer matrix composed of PCL:PET containing HEPES generated porosity. Diffusion is approximately 4-6 orders of magnitude slower through the PCL:PET matrix (˜) than through the hydrated pores (→). Clearly, higher HEPES contents allow for a longer effective pathway for “fast diffusion” and, therefore, a greater release rate.

Typical porogens (2,3,23,38) used for drug delivery are large and insoluble in the polymer matrices of interest. While this creates high interconnectivity between the resulting pores, release usually occurs in an undesirable biphasic fashion (23,38). However, in the current context, the incorporation of initially soluble salts into polymer blends ultimately achieves zero-order release and greater control over the release rate. In addition, while the RB release experiments ended after 10 days of water absorption, FIG. 2 shows that the different carrier oils can continue to hydrate to enable longer term release. Plateaus in water infusion are observed after ˜28 days of exposure regardless of carrier oil or porogen content, suggesting that release can continue to occur through the hydrated network of disconnected pores. By carefully varying the amount of the soluble salt species, tailorable release could be obtained for specific applications where varied drug release rates are desired.

### CONCLUSIONS

Incorporation of porogens into drug delivery systems is typically employed to achieve release rates that meet desired goals. In this work, we successfully incorporated a soluble salt (HEPES) into the polymer blend solution (PCL:PET) prior to electrospinning. Water treatment was used to remove the salt and introduce controlled porosity. Surprisingly, the mechanical properties of water-treated PCL:PET:HEPES compositions were generally higher than their PCL:PET counterparts. This is thought to be a result of the smaller fiber diameter (for as-spun samples) and the presence of residual sodium ions that enhance secondary bonding between polymer chains. Zero-order release of a model compound was observed for various PCL:PET:HEPES ratios. The release rate increased non-linearly versus initial HEPES concentration; COMSOL simulations support the idea that release is controlled by networks of hydrated discontinuous porosity in which elution occurs in a saltatory fashion. Overall, this study provides design guidance for the incorporation of soluble salts into thermolabile polymeric specimens that need to avoid high-temperature processes (i.e., melt extrusion) to achieve controllable, long-term release.

### REFERENCES

1. Zhang C, Chen X, Liu G, Chen R, Guo S. Mechanism and Kinetics of Drug Release from Poly(ε-Caprolactone) Based Extrudates Prepared by Hot-Melt Extrusion. J Macromol Sci Part B Phys. 2016; 55(3):285-98.
2. Lance K D, Good S D, Mendes T S, Ishikiriyama M, Chew P, Estes L S, et al. In vitro and in vivo sustained zero-order delivery of Rapamycin (sirolimus) from a biodegradable intraocular device. Investig Ophthalmol Vis Sci. 2015; 56(12):7331-7.
3. Yin H M, Qian J, Zhang J, Lin Z F, Li J S, Xu J Z, et al. Engineering porous poly(lactic acid) scaffolds with high mechanical performance via a solid state extrusion/porogen leaching approach. Polymers (Basel). 2016; 8(6).
4. Mane S. Effect of Porogens (Type and Amount) on Polymer Porosity: A Review. Can Chem Trans. 2016; 4(2):210-25.
5. Peng X, Mi H, Yu P, Qu J, Chen B, Jing X. Fabrication of Interconnected Porous Poly(lactic acid) Scaffolds Based on Dynamic Elongational Flow Procedure, Batch Foaming and Particulate Leaching. Mater Des. 2016; 04(01):220-4.
6. Tran R T, Naseri E, Kolasnikov A, Bai X, Yang J. A new generation of sodium chloride porogen for tissue engineering. Biotechnol Appl Biochem. 2011; 58(5):335-44.
7. Gupta A, Saquing C D, Afshan M, Tonelli A E, Khan S A, Kotek R. Porous nylon-6 fibers via a novel salt-induced electrospinning method. Macromolecules. 2009; 42(3):709-15.
8. Nam J, Huang Y, Agarwal S, Lannutti J. Improved Cellular Infiltration in Electrospun Fiber via Engineered Porosity. Tissue Eng. 2007 September; 13(9):2249-57.
9. Fredenberg S, Wahlgren M, Reslow M, Axelsson A. Pore formation and pore closure in poly(D,L-lactide-co-glycolide) films. J Control Release. Elsevier B. V.; 2011; 150(2):142-9.
10. Wang Y, Wang B, Wang G, Yin T, Yu Q. A novel method for preparing electrospun fibers with nano-/micro-scale porous structures. Polym Bull. 2009; 63(2):259-65.
11. Cho M, Kim S H, Jin G, Park K I, Jang J H. Salt-Induced Electrospun Patterned Bundled Fibers for Spatially Regulating Cellular Responses. ACS Appl Mater Interfaces. 2016; 8(21):13320-31.
12. Cazan C, Cosnita M, Duta A. Effect of PET functionalization in composites of rubber-PET-HDPE type. Arab J Chem. 2017; 10(3):300-12.
13. Meng X, Perry S L, Schiffman J D. Complex Coacervation: Chemically Stable Fibers Electrospun from Aqueous Polyelectrolyte Solutions. ACS Macro Lett. 2017; 6(5):505-11.
14. Matabola K P, Moutloali R M. The influence of electrospinning parameters on the morphology and diameter of poly(vinyledene fluoride) nanofibers—Effect of sodium chloride. J Mater Sci. 2013; 48(16):5475-82.
15. Chaparro F J, Presley K F, Coutinho da Silva M A, Mandan N, Colachis M L, Posner M, et al. Sintered Electrospun Polycaprolactone-Polyethylene Terephthalate for Drug Delivery. J Appl Polym Sci. 2019;
16. Chaparro F J, Presley K F, Coutinho da Silva M A, Lannutti J J. Sintered electrospun polycaprolactone for controlled model drug delivery. Mater Sci Eng C. 2019; 99.
17. Nam J, Huang Y, Agarwal S, Lannutti J. Materials Selection and Residual Solvent Retention in Biodegradable Electrospun Fibers. J Appl Polym Sci. 2008; 107:1547-54.
18. Drilling S, Gaumer J, Lannutti J. Fabrication of burst pressure competent vascular grafts via electrospinning: Effects of microstructure. J Biomed Mater Res—Part A. 2009; 88(4):923-34.
19. Gao Y, Teoh T W, Wang Q, Williams G R. Electrospun organic-inorganic nanohybrids as sustained release drug delivery systems. J Mater Chem B. Royal Society of Chemistry; 2017; 5(46):9165-74.
20. Werts M H V, Raimbault V, Texier-Picard R, Poizat R, Franais O, Griscom L, et al. Quantitative full-colour transmitted light microscopy and dyes for concentration mapping and measurement of diffusion coefficients in microfluidic architectures. Lab Chip. 2012; 12(4):808-20.
21. R Core Team. R: A language and environment for statistical computing. Vienna, Austria: R Foundation for Statistical Computing; 2013.
22. Annabi N, Fathi A, Mithieux S M, Weiss A S, Dehghani F. Fabrication of porous PCL/elastin composite scaffolds for tissue engineering applications. J Supercrit Fluids. Elsevier B. V.; 2011; 59:157-67.
23. Webber W L, Lago F, Thanos C, Mathiowitz E. Characterization of soluble salt-loaded, degradable PLGA films and their release of tetracycline. J Biomed Mater Res. 1998; 41(1):18-29.
24. Barbanti S H, Carvalho Zavaglia C A, De Rezende Duek E A. Effect of Salt Leaching on PCL and PLGA (50/50) Resorbable Scaffolds. Mater Res. 2008; 11(1):75-80.
25. Seo E H, Na K. Polyurethane membrane with porous surface for controlled drug release in drug eluting stent. 2014; 14-8.
26. Harrison K L, Jenkins M J. The effect of crystallinity and water absorption on the dynamic mechanical relaxation behaviour of polycaprolactone. Polym Int. 2004; 53(9):1298-304.
27. Williams, GR; Chatterton, NP; Nazir, T; Yu, DG; Zhu, LM; Branford-White C. Electrospun nanofibers in drug delivery: recent developments and perspectives. Ther Deliv. 2012; 3(4):515-33.
28. Fahr A, Liu X. Drug delivery strategies for poorly water-soluble drugs. Expert Opin Drug Deliv. 2007; 4(4):403-16.
29. Zaitseva I S, Sytnik O Y, Krasnoperova A P, Bondarev N V. Influence of properties of nonaqueous solvents on the solubility of NH4Cl and thermodynamics of its solution. Russ J Gen Chem. 2005; 75(1):25-30.
30. Cengiz F, Jirsak O. The effect of salt on the roller electrospinning of polyurethane nanofibers. Fibers Polym. 2009; 10(2):177-84.
31. Baicu S C, Taylor M J. Acid-base buffering in organ preservation solutions as a function of temperature: New parameters for comparing buffer capacity and efficiency. Cryobiology. 2002; 45(1):33-48.
32. Fredenberg S, Reslow M, Axelsson A. Effect of divalent cations on pore formation and degradation of poly(D,L-lactide-co-glycolide). Pharm Dev Technol. 2007; 12(6):563-72.
33. Chaparro F J. Biocompatible Electrospun Vehicles To Enhance the Effectiveness Of Anti-Fertility Strategies And Their Biomimetic Properties As Blood Vessel Scaffolds [Internet]. [Columbus, OH]: The Ohio State University; 2018 [cited 2018 Sep. 5]. Available from: http://rave.ohiolink.edu/etdc/view?acc_num=osu1514986344784852
34. Liu H, Leonas K K, Zhao Y. Antimicrobial Properties and Release Profile of Ampicillin from Electrospun Poly (ε-caprolactone) Nanofiber Yarns. J Eng Fiber Fabr. 2010; 5(4):10-9.
35. Valdes Garcia A, Ramos Santonja M, Sanahuja A B, Del Carmen Garrigos Selva M. Characterization and degradation characteristics of poly(ε-caprolactone)-based composites reinforced with almond skin residues. Polym Degrad Stab. Elsevier Ltd; 2014; 108:269-79.
36. Wong S C, Baji A, Leng S. Effect of fiber diameter on tensile properties of electrospun poly(ε-caprolactone). Polymer (Guildf). 2008; 49(21):4713-22.
37. Bleach N C, Nazhat S N, Tanner K E, Kellomaki M, Tormala P. Effect of filler content on mechanical and dynamic mechanical properties of particulate biphasic calcium phosphate-polylactide composites. Biomaterials. 2002; 23(7):1579-85.
38. Douglas P, Andrews G, Jones D, Walker G. Analysis of in vitro drug dissolution from PCL melt extrusion. Chem Eng J. Elsevier B. V.; 2010; 164(2-3):359-70.
39. Huxley A F, Stampfli R. Evidence for saltatory conduction in peripheral myelinated nerve fibres. J Physiol. 1949; 108(3):315-39.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A composition for drug delivery comprising:
a hydrophilic active agent encapsulated within a polysiloxane polymer;
wherein the polysiloxane polymer comprises a hydrophilic polysiloxane polymer, an amphiphilic polysiloxane polymer, or any combination thereof.

2. The composition of claim 1, wherein the hydrophilic active agent comprises a gonodotropin releasing hormone (GnRh) agonist, antagonist, bioconjugate or pharmaceutically acceptable salts or prodrugs, thereof such as deslorelin, histrelin, avorelin, leuprolide, triptorelin, nafarelin, goserelin, buserelin, or fertirelin; or an immunocontraceptive agent based on zona pellucida (ZP) (i.e., porcine zona pellucida) or gonadotropin-releasing hormone (GnRH); or
GnRH-based bioconjugates.

3. The composition of claim 1, wherein the polysiloxane polymer further comprises a hydrophobic polysiloxane polymer.

4. The composition of claim 1, wherein the hydrophilic active agent comprises a solid.

5. The composition of claim 1, wherein the gonodrotropin releasing hormone agonist comprises deslorelin or pharmaceutically acceptable salt or prodrug thereof.

6. The composition of claim 1, wherein the hydrophilic active agent is present in the composition at a concentration of from 1 μg/ml to 100,000 μg/ml.

7. A drug delivery capsule comprising:
two closed ends, a wall coaxially disposed around a chamber, and the composition defined by claim 1 disposed within the chamber;
wherein the wall comprises a biodegradable polymer.

8. The capsule of claim 7, wherein the wall further comprises a non-biodegradable polymer.

9. The capsule of claim 8, wherein the non-biodegradable polymer comprises polyethylene terephthalate (PET).

10. The capsule of claim 7, wherein the wall comprises a plurality of pores formed therewithin, wherein the pores have an average pore size of from 100 nm to 5 μm.

11. The capsule of claim 7, wherein the capsule has a length of from 0.1 cm to 5 cm.

12. The capsule of claim 7, wherein the chamber has a cross-sectional diameter of from 100 μm to 5000 μm.

13. The capsule of claim 7, wherein the wall has a thickness of from 1 μm to 1,000 μm.

14. The capsule of claim 7, wherein the active agent is present in the composition in an amount of from 1 μg/ml to 100,000 μg/ml.

15. The capsule of claim 7, wherein the capsule releases the hydrophilic active agent over a period of at least 30 days, at least 3 months, at least 6 months, at least 9 months, or at least 12 months when incubated in phosphate buffered saline (PBS) at 37° C.

16. The capsule of claim 7, wherein the biodegradable polymer comprises a polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof.

17. (canceled)

18. A method for preparing a drug delivery capsule, the method comprising:
electrospinning a wall forming solution comprising a biodegradable polymer and a porogen to form a wall precursor having a lumen extending therethrough from a first end to a second end;
removing the porogen from the wall precursor;
sintering the wall precursor;
injecting the composition from claim 1 into the lumen of the wall precursor; and
sealing the first end and the second end to form the drug delivery capsule.

19. The method of claim 18, wherein electrospinning the wall forming solution comprises:
electrically charging the wall forming solution; and
discharging the electrically charged wall forming solution onto a grounded target under an electrostatic field, such that the movement of the electrically charged wall forming solution under the electrostatic field causes the electrically charged wall forming solution to evaporate and produce fibers that form the wall precursor on the grounded target.

20. The method of claim 19, wherein the grounded target comprises a rotating mandrel.

21. The method of claim 18, wherein the porogen comprises an organic salt.

22. (canceled)

23. The method of claim 18, wherein the biodegradable polymer comprises polyester, polylactic acid (PLA), polyglycolic acid (PGA), polyethylene oxide (PEO), poly lactic-co-glycolide (PLGA), polycaprolactone (PCL), polydioxanone (PDS), a polyhydroxyalkanoate (PHA), polyurethane (PU), a poly(phosphazine), a poly(phosphate ester), a gelatin, a collagen, a polyethylene glycol (PEG), gelatin, collagen, elastin, silk fibroin, copolymers thereof, and blends thereof.

24. (canceled)

25. The method of claim 18, wherein the wall forming solution further comprises a non-biodegradable polymer.

26. (canceled)

27. A method of contraceptive treatment in a subject in need thereof comprising:
administering to the subject an effective amount of an active agent using a drug delivery capsule of claim 7.
